(19) United States
(12) Patent Application Publication
(10) Pub. No.: US 2025/0226094 A1
(43) Pub. Date: Jul. 10, 2025

(54) SYSTEMS AND METHODS FOR PHARYNGEAL PHENOTYPING IN OBSTRUCTIVE SLEEP APNEA

(71) Applicant: VANDERBILT UNIVERSITY, Nashville, TN (US)

(72) Inventors: Daniel FABBRI, Nashville, TN (US); David T. KENT, Nashville, TN (US); Cheng YE, Nashville, TN (US)

(21) Appl. No.: 18/853,062

(22) PCT Filed: Mar. 31, 2023
(86) PCT No.: PCT/US2023/017137

§ 371 Date: Sep. 30, 2024

Related U.S. Application Data
(60) Provisional application No. 63/326,322, filed on Apr. 1, 2022.

(51) Int. Cl. G16H 50/20; G06N 20/10; G16H 10/60; G16H 20/40; G16H 30/40; G16H 50/50

(52) U.S. Cl. CPC G16H 50/20; G06N 20/10; G16H 10/60; G16H 20/40; G16H 30/40; G16H 50/50

(57) ABSTRACT

Systems and methods for pharyngeal phenotyping in obstructive sleep apnea are described herein. An example method includes receiving manometry data for a subject; extracting a plurality of features from the manometry data, where the extracted features include one or more of a high-level breath feature, a frequency feature, or a largest negative connected component (LNCC) feature; inputting the extracted features into a trained machine learning model; and predicting, using the trained machine learning model, at least one of a location of pharyngeal collapse for the subject or a degree of pharyngeal collapse for the subject.

### CROSS-REFERENCE TO RELATED APPLICATIONS

This application claims the benefit of U.S. provisional patent application No. 63/326,322, filed on Apr. 1, 2022, and titled “SYSTEMS AND METHODS FOR PHARYNGEAL PHENOTYPING IN OBSTRUCTIVE SLEEP APNEA,” the disclosure of which is expressly incorporated herein by reference in its entirety.

### BACKGROUND

Obstructive sleep apnea (OSA) is a common sleep disorder characterized by repetitive upper airway collapse that has been independently associated with hypertension, cardiovascular disease risk, and decreased work productivity.¹⁻⁵ Positive airway pressure (PAP) can be an efficacious first-line treatment for OSA, but effectiveness can be compromised by non-compliance, with only 39% to 50% of patients maintaining adequate usage.⁶⁻⁸ The American Academy of Sleep Medicine recommends that adults with OSA who are unaccepting or intolerant of PAP and with a body mass index less than 40 kg/m² be given the option to consult with a surgeon regarding surgical interventions for OSA.⁹

Surgical procedural selection for OSA can be a challenging task for even the experienced surgeon. Drug-induced sleep endoscopy (DISE) was first described in 1991 as a diagnostic tool to aid surgical procedural selection and has more recently found favor in the US.¹⁰ During a DISE examination, patients are pharmacologically sedated and a trans-nasal flexible fiberoptic endoscope is used to observe dynamic collapse patterns of the upper airway. The Food and Drug Administration requires DISE examination to determine eligibility for hypoglossal nerve stimulation therapy, as patients with complete circumferential collapse of the soft palate (CCC) are considered less likely to respond to the treatment.¹¹ Despite its growing popularity, DISE has limitations. Evaluation techniques have not been standardized, and multiple anesthetic agents and pharyngeal classification systems have been described.^{10, 12-17} DISE is expensive, requires sedation, only approximates natural sleep, and inter- and intra-rater assessments of collapse sites and patterns are variable.¹⁸⁻²³ Other imaging modalities including x-ray, ultrasound, computed tomography, and magnetic resonance imaging have been investigated but have not found widespread favor due to various intrinsic limitations such as cost, lack of dynamic assessment, and difficulty approximating natural sleep conditions.

Manometry is a diagnostic study assessing pressure changes within the lumen of a hollow organ using one or more pressure sensors. Esophageal manometry has long been considered the gold standard for detection of respiratory effort during polysomnography, although it is now rarely employed in clinical sleep practice.²⁴ Conversely, over the past decade high-resolution esophageal manometry, with multiple solid-state pressure sensors at closely spaced intervals, has become the gold standard for assessment of esophageal motility disorders.²⁵ To access the esophagus, a catheter with pressure sensors is passed through the nose and pharynx. If the sensors remain in the pharynx and are not passed into the esophagus, measurements of pharyngeal pressure changes instead become possible. Conventional pharyngeal manometry was investigated as early as the 1970s.²⁶ In 1992, Woodson and Wooten investigated its use for surgical phenotyping of the upper airway, with the authors reporting that it enabled comparison of simultaneous events at multiple pharyngeal sites with minimal disruption of sleep.^{27, 28} Nevertheless, use of this technique for dynamic pharyngeal assessment in sleep can be limited by equipment costs, the unintuitive nature of the manometry waveforms, and the high volume of data requiring labor-intensive analysis.

Therefore what is needed are improvements that address these and other concerns.

### SUMMARY

An example method for pharyngeal phenotyping in obstructive sleep apnea is described herein. The method includes receiving manometry data for a subject; extracting a plurality of features from the manometry data, where the extracted features include one or more of a high-level breath feature, a frequency feature, or a largest negative connected component (LNCC) feature; inputting the extracted features into a trained machine learning model; and predicting, using the trained machine learning model, at least one of a location of pharyngeal collapse for the subject or a degree of pharyngeal collapse for the subject.

In some implementations, the high-level breath feature includes a measure of manometry values across a plurality of pressure sensors of a manometry instrument.

In some implementations, the frequency feature includes a measure of a rate of change of manometry values per pressure sensor of a manometry instrument over the course of a breath.

In some implementations, the LNCC feature includes a measure of a shape or

structure of a negative pressure envelop within an inspiration across a plurality of pressure sensors of a manometry instrument.

In some implementations, the location of pharyngeal collapse and the degree of pharyngeal collapse are predicted using the trained machine learning model.

In some implementations, the location of pharyngeal collapse is defined by a level of pharyngeal collapse. Optionally, the level of pharyngeal collapse is the subject's velum, oropharynx, or hypopharynx. Alternatively or additionally, the location of pharyngeal collapse is defined by an anatomic structure. Optionally, the anatomic structure is the subject's soft palate, oropharyngeal lateral walls, tongue base, or epiglottis.

In some implementations, the degree of pharyngeal collapse is defined by a pharyngeal phenotyping classification system. Optionally, the pharyngeal phenotyping classification system is the VOTE (velum, oropharynx, tongue base, or epiglottis) classification system.

In some implementations, the method includes receiving patient data associated with the subject; and extracting one or more features from the patient data, where the extracted features include the plurality of features extracted from the manometry data and the one or more features extracted from the patient data. Optionally, the patient data includes anthropomorphic data, demographic data, polysomnography (PSG) data, video data, or electronic medical record data.

In some implementations, the method includes generating display data for the location of pharyngeal collapse. Optionally, the method includes presenting the display data on a display device. Alternatively or additionally, the method includes generating a text-based description of the location of pharyngeal collapse. Optionally, the method includes presenting the text-based description on a display device.

In some implementations, the method includes generating a heatmap image associated with a breath from the manometry data. Optionally, the method includes presenting the heatmap image on a display device. Optionally, presenting the heatmap image on a display device includes selecting a representative breath from the manometry data and a corresponding heatmap image associated with the representative breath; and presenting the corresponding heatmap image on a display device.

In some implementations, the trained machine learning model is a supervised machine learning model. In some implementations, the trained machine learning model is a semi-supervised learning model.

In some implementations, the trained machine learning model is a K-nearest neighbor (k-NN) classifier.

In some implementations, the trained machine learning model is a support vector machine classifier, a logistic regression classifier, a random forest classifier, or a Naïve Bayes classifier.

An example method for treating a subject with obstructive sleep apnea is also described herein. The method includes performing pharyngeal phenotyping as described herein. Additionally, the method includes recommending the subject for an intervention based on the at least one of the location of pharyngeal collapse or the degree of pharyngeal collapse predicted by the trained machine learning model. Optionally, the method further includes performing the intervention on the subject. Alternatively or additionally, the intervention includes implanting a medical device in the subject, the medical device being configured to deliver nerve stimulation therapy. Alternatively or additionally, the intervention is a surgical, medical, or device-based intervention.

An example method for predicting whether a subject is a favorable or non-favorable candidate for an intervention is also described herein. The method includes receiving manometry data for a subject; extracting a plurality of features from the manometry data for the subject, where the extracted features include one or more of a high-level breath feature, a frequency feature, or a largest negative connected component (LNCC) feature; inputting the extracted features into a trained machine learning model; and predicting, using the trained machine learning model, whether the subject is a favorable or non-favorable candidate for an intervention.

An example system for pharyngeal phenotyping in obstructive sleep apnea is also described herein. The system includes at least one processor; and a memory operably coupled to the at least one processor, the memory having computer-executable instructions stored thereon. The processor is configured to receive manometry data for a subject; extract a plurality of features from the manometry data for the subject, where the extracted features include one or more of a high-level breath feature, a frequency feature, or a largest negative connected component (LNCC) feature; input the extracted features into a trained machine learning model; and receive, from the machine learning model, at least one of a location of pharyngeal collapse for the subject or a degree of pharyngeal collapse for the subject.

In some implementations, the high-level breath feature includes a measure of manometry values across a plurality of pressure sensors of a manometry instrument.

In some implementations, the frequency feature includes a measure of a rate of change of manometry values per pressure sensor of a manometry instrument over the course of a breath.

In some implementations, the LNCC feature includes a measure of a shape or structure of a negative pressure envelop within an inspiration across a plurality of pressure sensors of a manometry instrument.

In some implementations, the location of pharyngeal collapse and the degree of pharyngeal collapse are predicted using the trained machine learning model.

In some implementations, the location of pharyngeal collapse is defined by a level of pharyngeal collapse. Optionally, the level of pharyngeal collapse is the subject's velum, oropharynx, or hypopharynx. Alternatively or additionally, the location of pharyngeal collapse is defined by an anatomic structure. Optionally, the anatomic structure is the subject's soft palate, oropharyngeal lateral walls, tongue base, or epiglottis.

In some implementations, the degree of pharyngeal collapse is defined by a pharyngeal phenotyping classification system.

In some implementations, the processor is further configured to receive patient data associated with the subject; and extract one or more features from the patient data, where the extracted features include the plurality of features extracted from the manometry data and the one or more features extracted from the patient data. Optionally, the patient data includes anthropomorphic data, demographic data, polysomnography (PSG) data, video data, or electronic medical record data.

In some implementations, the processor is further configured to generate display data for the location of pharyngeal collapse. Optionally, the processor is further configured to present the display data on a display device. Alternatively or additionally, the processor is further configured to generate a text-based description of the location of pharyngeal collapse. Optionally, the processor is further configured to present the text-based description on a display device.

In some implementations, the processor is further configured to generate a heatmap image associated with a breath from the manometry data. Optionally, the processor is further configured to present the heatmap image on a display device. Optionally, presenting the heatmap image on a display device includes selecting a representative breath from the manometry data and a corresponding heatmap image associated with the representative breath; and presenting the corresponding heatmap image on a display device.

In some implementations, the trained machine learning model is a supervised machine learning model. In some implementations, the trained machine learning model is a semi-supervised learning model.

In some implementations, the trained machine learning model is a K-nearest neighbor (k-NN) classifier.

In some implementations, the trained machine learning model is a support vector machine classifier, a logistic regression classifier, a random forest classifier, or a Naïve Bayes classifier.

An example method for training a machine learning model for pharyngeal phenotyping in obstructive sleep apnea is also described herein. The method includes receiving respective manometry data for each of a plurality of patients; receiving identification of a plurality of flow-limited breaths; receiving a respective location of pharyngeal collapse for each of the identified flow-limited breaths; and receiving a respective degree of pharyngeal collapse for each of the identified flow-limited breaths. The method also includes creating a training dataset including the respective manometry data for each of the patients, the respective location of pharyngeal collapse for each of the identified flow-limited breaths, and the respective degree of pharyngeal collapse for each of the identified flow-limited breaths; and training a machine learning model using the training dataset. The trained machine learning model is configured to predict at least one of a location of pharyngeal collapse for a subject or a degree of pharyngeal collapse for the subject.

In some implementations, the training dataset includes a plurality of features associated with the respective manometry data for each of the patients, the features including high-level breath features, frequency features, and largest negative connected component (LNCC) features. Optionally, the method includes performing feature selection to select a set of predictive features among the plurality of features.

In some implementations, the method includes augmenting the training dataset to include a plurality of synthetic manometry data samples. Alternatively or additionally, the training dataset further includes respective patient data for the plurality of patients, the respective patient data including anthropomorphic data, demographic data, polysomnography (PSG) data, video data, or electronic medical record data.

An example system for identifying a location or locations of pharyngeal collapse is described herein. The system includes at least one processor; and a memory operably coupled to the at least one processor, the memory having computer-executable instructions stored thereon. The processor is configured to receive manometry data for a subject; analyze the manometry data to track a nadir position over time in the subject's airway; and identify a location of pharyngeal collapse for the subject using the nadir position over time in the subject's airway.

In some implementations, the system further includes a manometry instrument having a plurality of pressure sensors. The manometry instrument can be operably coupled to the at least one processor and memory. The manometry data is measured by the manometry instrument and received by the at least one processor.

In some implementations, the memory has further computer-executable instructions stored thereon that, when executed by the at least one processor, cause the at least one processor to extract a plurality of features from the manometry data, where the extracted features are analyzed to track the nadir position over time in the subject's airway.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIGS. 1A-1B illustrate block diagrams of implementations of the present disclosure.

FIG. 1A illustrates a method of performing a prediction based on manometry data using a trained machine learning model (i.e., inference mode) according to one implementation of the present disclosure; FIG. 1B illustrates a method of training the machine learning model shown in FIG. 1A according to one implementation of the present disclosure.

FIG. 2 is an example computing device.

FIG. 3 illustrates a high-resolution manometry catheter that is 6-French (2 mm) and comprised of 21 solid-state sensors spaced at 8 mm intervals, except for a 6 cm gap between the first and second most distal sensors to aid anchoring it in the esophagus.

FIG. 4 illustrates an example image visualizing high-resolution manometry data (bottom of FIG. 4) simultaneously with high-resolution endoscopy video (top of FIG. 4). Time is on the x-axis, with anatomic position of each sensor represented from superior to inferior on the y-axis. Manometry data is represented as a heatmap, with a color scale 400 seen at the left side of the image. Atmospheric pressure at the warm (red) end 402 of the spectrum transitioned to cooler (blue) colors at the most negative recorded values at the opposite end 404. The constant positive pressures of the upper esophageal sphincter (UES) 406 skewed into violet colors.

FIG. 5 illustrates a subset of high-resolution manometry heatmaps representing each of the four dominant sites of collapse under the VOTE classification, using the same legend of heatmap construction as FIG. 4. V=velum; O=oropharynx; T=tongue base; E=epiglottis.

FIGS. 6A-6B illustrates an example of stepwise conversion of a high-resolution manometry heatmap (FIG. 6A) to the largest negative connected component (LNCC) (FIG. 6B). A LNCC represents the dominant shape of the negative pressure envelope within an inspiration.

FIG. 7 illustrates a table of mean demographic variables for participants included in final analyses of the example below. Standard deviations are given in parenthesis. There were no significant differences in demographic or polysomnographic variables between included and excluded participants. BMI=body mass index; AHI=apnea-hypopnea index.

FIG. 8 illustrates the distribution of airway collapse patterns observed across the 40 included participants using the VOTE classification system.

FIG. 9 illustrates a table of Area Under the Curve and F1-scores from the best model predicting pharyngeal structural collapse based only on high-resolution manometry data. The holdout test set (n=9) did not contain any E=1 values. V=velum, O=oropharynx, T=tongue base, E=epiglottis.

FIG. 10 illustrates example of high-resolution manometry data from partial anterior-posterior velopharyngeal collapse. Sensors (horizontal white lines) record pressure (mm Hg; color scale) at 100 Hz (time; x-axis) at 8 mm intervals within the pharynx (y-axis).

FIG. 11 illustrates example of high-resolution manometry data from partial oropharyngeal collapse. Sensors (horizontal white lines) record pressure (mm Hg; color scale) at 100 Hz (time; x-axis) at 8 mm intervals within the pharynx (y-axis).

FIG. 12 illustrates example of high-resolution manometry data from complete tongue base collapse. Sensors (horizontal white lines) record pressure (mm Hg; color scale) at 100 Hz (time; x-axis) at 8 mm intervals within the pharynx (y-axis).

FIG. 13 illustrates example of high-resolution manometry data from complete epiglottic collapse. Sensors (horizontal white lines) record pressure (mm Hg; color scale) at 100 Hz (time; x-axis) at 8 mm intervals within the pharynx (y-axis).

FIGS. 14A-14B illustrate combined endoscopy (FIG. 14A) and high-resolution manometry (FIG. 14B). In the first and third breath, palatal collapse (black arrowheads 1402 in FIG. 14B) precedes secondary and more negative hypopharyngeal collapse later in the breath. The second breath demonstrates synchronous palatal and hypopharyngeal collapse (white arrowhead 1404 in FIG. 14B).

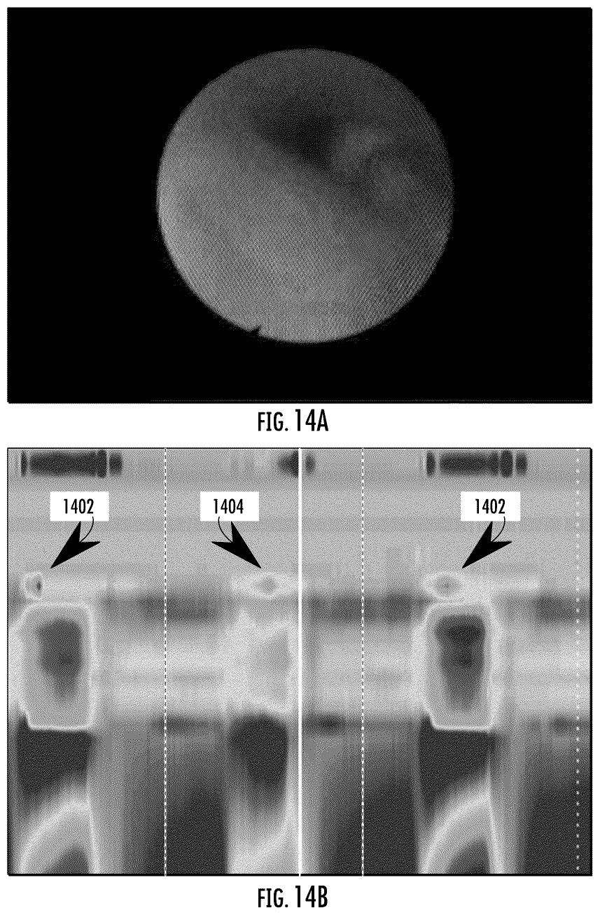

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “about” or “approximately” when referring to a measurable value such as an amount, a percentage, and the like, is meant to encompass variations of ±20%, ±10%, ±5%, or ±1% from the measurable value.

“Administration” of “administering” to a subject includes any route of introducing or delivering to a subject an agent. Administration can be carried out by any suitable means for delivering the agent. Administration includes self-administration and the administration by another.

The term “subject” is defined herein to include animals such as mammals, including, but not limited to, primates (e.g., humans), cows, sheep, goats, horses, dogs, cats, rabbits, rats, mice and the like. In some embodiments, the subject is a human.

Drug-induced sleep endoscopy (DISE) is a commonly used diagnostic tool for surgical procedural selection in OSA, but it can be expensive, subjective, and require sedation. Thus, systems and methods for upper airway phenotyping in OSA, including machine-learning based prediction methods and training methods, are described herein. Such systems and methods can reliably predict pharyngeal sites of collapse based solely on manometric recordings. Additionally such systems and methods address challenges presented by conventional DISE examinations and/or analysis of manometry data. For example, the systems and methods described herein may provide an alternative to DISE examination, which is currently required for determining eligibility for certain surgical interventions. DISE examination has limitations including, but not limited to, lacking standardization, requiring sedation, and high costs. The systems and methods described herein may also provide a means to make predictions about pharyngeal collapse and/or patient outcome based solely on the patient's manometry data. Esophageal manometry has limitations including, but not limited to, difficulties interpreting waveforms, voluminous data (which require dedicated labor and computing resources), and high costs. The systems and methods described herein address challenges of conventional techniques at least in part using machine learning to analyze and make predictions from complex manometry data. Additionally, merely inputting raw manometry data (as is) into a supervised machine learning does not produce highly accurate results. In contrast, the systems and methods described herein, which involve extraction of high-level features based on the subject's breath and structure of collapse (e.g., high-level breath features, frequency features, LNCC features), provide for accurate predictions. The machine learning model training methods described herein are designed to enable accurate predictions based on unique nature of manometry data. Accordingly, the machine learning based manometry analysis described herein can complement standard of care sleep studies and/or other diagnostic studies (imaging or otherwise) designed to ascertain the structure of the upper airway and the vulnerability of different components to collapse during sleep.

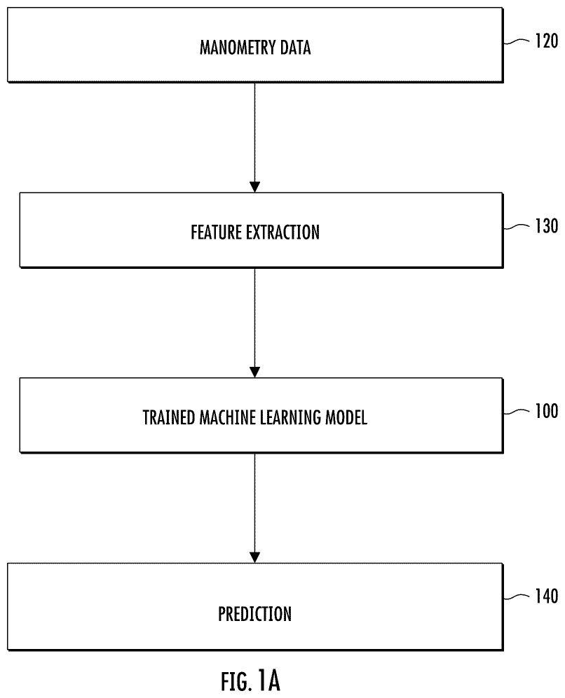

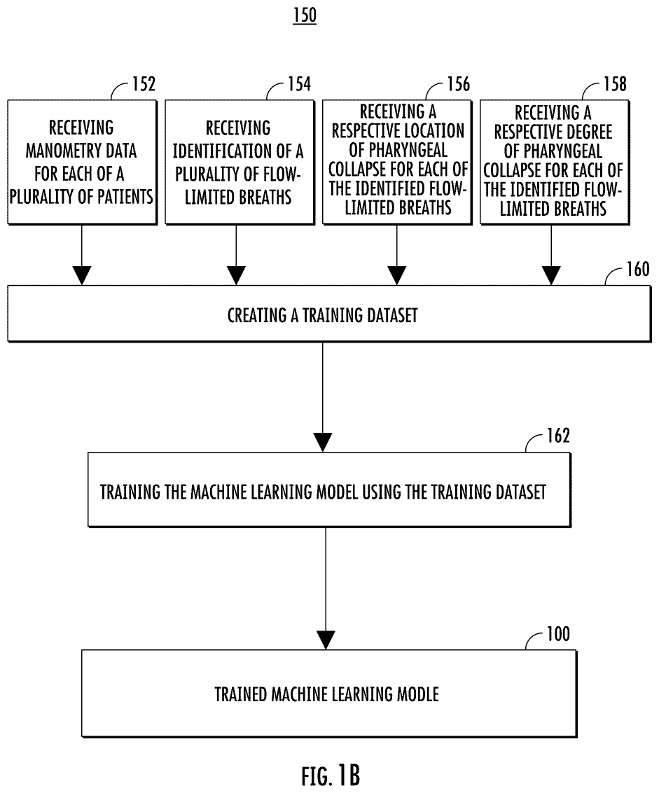

Referring now to FIG. 1A, a block diagram illustrating use of a trained machine learning model 100 in inference mode is shown. In FIG. 1A, the machine learning model is a trained machine learning model. As described in more detail below, techniques for machine learning model training are discussed in greater detail below. it should be understood that a trained machine learning model 100 maps an input (sometimes referred to as “features”) to an output (sometimes referred to as “target” or “targets”). As described herein, the input may be manometry data 120 and/or one or more features 130 extracted from manometry data, and the output may be a prediction 140 of location of pharyngeal collapse for the subject or degree of pharyngeal collapse for the subject. Optionally, in some implementations, the output may be a prediction 140 of both location and degree of pharyngeal collapse for the subject. Alternatively or additionally, in some implementations, the output may be a prediction 140 of whether a subject is a favorable or unfavorable candidate for an intervention. This disclosure contemplates that the intervention may be a medical or surgical intervention. Medical interventions can include treatment regimens or protocols. For example, medical interventions may be administration of a therapeutic agent or delivery of non-invasive treatment. Surgical interventions can include implantation of a medical device configured to deliver neural stimulation. In some implementations, this disclosure contemplates that the machine learning model 100 outputs a probability (e.g., logistic regression). In other implementations, this disclosure contemplates that the machine learning model performs a classification (e.g., k-NN classification).

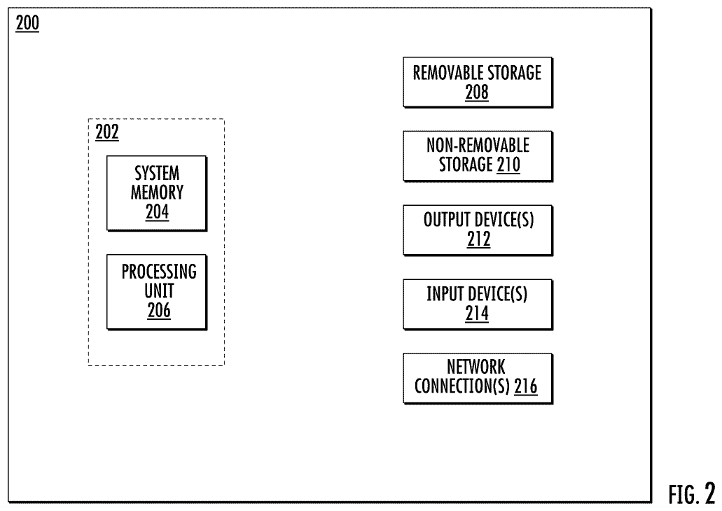

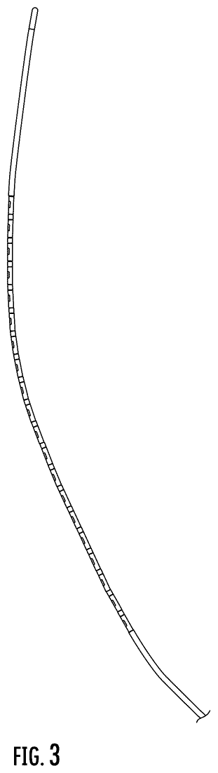

Still with reference to FIG. 1A, an implementation of the present disclosure is shown including a method for pharyngeal phenotyping in obstructive sleep apnea. The manometry data 120 is received, for example, by a computing device such as the computing device shown in FIG. 2. Manometry data can be acquired using a manometry catheter. Manometry catheters are known in the art. Example manometry catheters are commercially available, for example, from Unisensor AG of Zurich, Switzerland. It should be understood that Unisensor AG is provided only as an examples source for manometry catheters. Additionally, a plurality of features 130 are optionally extracted from the manometry data 120. The features 130 extracted can include one or more high-level breath features. In some implementations, a high-level breath feature is a measure of manometry values across a plurality of pressure sensors of a manometry instrument (see e.g., FIG. 3). Alternatively or additionally, the features 130 extracted can include one or more frequency features. In some implementations, a frequency feature is a measure of a rate of change of manometry values per pressure sensor of a manometry instrument (see e.g., FIG. 3) over the course of a breath. Alternatively or additionally, the features 130 extracted can include one or more largest negative connected component (LNCC) features. In some implementations, a LNCC feature is a measure of a shape or structure of a negative pressure envelop within an inspiration across a plurality of pressure sensors of a manometry instrument (see e.g., FIG. 3). High-level breath features, frequency features, and LNCC features are described in detail in the Examples below. Optionally, the manometry instrument is a pharyngeal and esophageal manometry instrument. In the examples described herein, the pressure sensors used to take measurements are mostly located in the subject's pharynx, while a few span the upper esophageal sphincter and/or located in the esophagus. It should be understood that the number and arrangement of pressure sensors in the manometry instrument provided in the examples are only provided as examples. This disclosure contemplates using manometry instruments having different numbers and/or arrangements of pressure sensors. Additionally, this disclosure contemplates that the manometry instrument and/or the pressure sensors can be operably coupled to the computing device, for example, using one or more communication links. This disclosure contemplates the communication links are any suitable communication link. For example, a communication link may be implemented by any medium that facilitates data exchange including, but not limited to, wired, wireless and optical links.

It should be understood that high-level breath features, frequency features, and LNCC features are provided only as examples. This disclosure contemplates that features 130 may include one or more features extracted from patient data. Patient data can include, but is not limited to, anthropomorphic data (e.g., height, weight, body mass index (BMI), neck/hip/waste circumference, tonsil size, tongue size, etc.), demographic data (e.g., race, age, gender, etc.), polysomnography (PSG) data (e.g., electroencephalography (EEG), electrooculography (EOG), electromyography (EMG), electrocardiogram (ECG), pulse oximetry (Sp0₂), respiratory inductance plethysmography, etc.), position sensor data, other sensor data like non-invasive airflow data (e.g., nasal pressure transducer, oronasal thermistor, pneumotachometry, positive airway pressure, or other biophysiologic time series data), video data, and electronic medical record data. As described above, the machine learning model 100 is a trained and can output a prediction 140 based on the extracted features 130. In some implementations, the prediction 140 is a location of pharyngeal collapse for the subject. Alternatively or additionally, in some implementations, the prediction 140 is a degree of pharyngeal collapse for the subject. Alternatively or additionally, in some implementations, the prediction 140 is whether a subject is a favorable or unfavorable candidate for an intervention.

In one implementation described herein, the machine learning model 100 is a K-nearest neighbor (k-NN) classifier. A k-NN classifier is a supervised classification model that classifies new data points based on similarity measures (e.g., distance functions). k-NN classifiers are trained with a dataset (also referred to herein as a “data set”) to maximize or minimize an objective function, for example a measure of the k-NN classifier's performance (e.g., error such as L1 or L2 loss), during training. This disclosure contemplates that any algorithm that finds the maximum or minimum of the objective function can be used. k-NN classifiers are known in the art and are therefore not described in further detail herein.

It should be understood that k-NN classifiers are provided only as an example. This disclosure contemplates using other supervised learning models with the systems and methods described herein. According to supervised learning, the model learns a function that maps an input (also known as feature or features) to an output (also known as target or target) during training with a labeled data set (or dataset). For example, as described herein, the labeled dataset may be manometry data for a plurality of subjects, where the manometry data includes labels for location of pharyngeal collapse and/or degree of pharyngeal collapse. It should be understood that location and degree of pharyngeal collapse are only provided as example “labels” in the manometry data. Non-limiting examples of supervised learning models that can be used with the systems and methods described herein include, but are not limited to, a support vector machine classifier, a logistic regression classifier, a random forest classifier, or a Naïve Bayes classifier, all of which are well-known in the art and therefore not described in further detail herein. Alternatively, this disclosure contemplates using semi-supervised learning models with the systems and methods described herein. According to semi-supervised learning, the model learns a function that maps an input (also known as feature or features) to an output (also known as target or target) during training with both labeled and unlabeled data. In this implementation, the dataset may be manometry data for a plurality of subjects, where some of the manometry data includes labels for location of pharyngeal collapse and/or degree of pharyngeal collapse and some of the manometry data is unlabeled. It should be understood that location and degree of pharyngeal collapse are only provided as example “labels” in the manometry data. Thus, this disclosure contemplates leveraging a larger dataset (i.e., including both labeled and unlabeled data) to train a semi-supervised learning model.

Alternatively, this disclosure contemplates using a deep learning model (e.g., an artificial neural network (ANN)) with the systems and methods described herein. An ANN is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanH, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include, but are not limited to, backpropagation.

As described above, in some implementations, the trained machine learning model 100 can output a prediction 140 of location of pharyngeal collapse for the subject. The location of pharyngeal collapse can be defined by a level of pharyngeal collapse, for example, the subject's velum, oropharynx, hypopharynx, or other level. Alternatively or additionally, the location of pharyngeal collapse can be defined by an anatomic structure, for example, the subject's soft palate, oropharyngeal lateral walls (which include the palatine tonsils (if present)), tongue base, epiglottis, or other structure. It should be understood that defining a location of pharyngeal collapse by level or anatomic structure, as well as specific examples thereof, are provided only as examples. This disclosure contemplates that the location of pharyngeal collapse may be defined by other means. Alternatively or additionally, in some implementations, the trained machine learning model 100 can output a prediction 140 of degree of pharyngeal collapse for the subject. The degree of pharyngeal collapse can be defined by a pharyngeal phenotyping classification system. A non-limiting example of a pharyngeal phenotyping classification system that can be used is the VOTE (velum, oropharynx, tongue base, or epiglottis) classification system (see e.g., the Examples described below). It should be understood that defining a degree of pharyngeal collapse using the VOTE classification system is provided only as an example. This disclosure contemplates that the degree of pharyngeal collapse may be defined by other means. Optionally, in some implementations, the trained machine learning model 100 can output a prediction 140 of both location and degree of pharyngeal collapse.

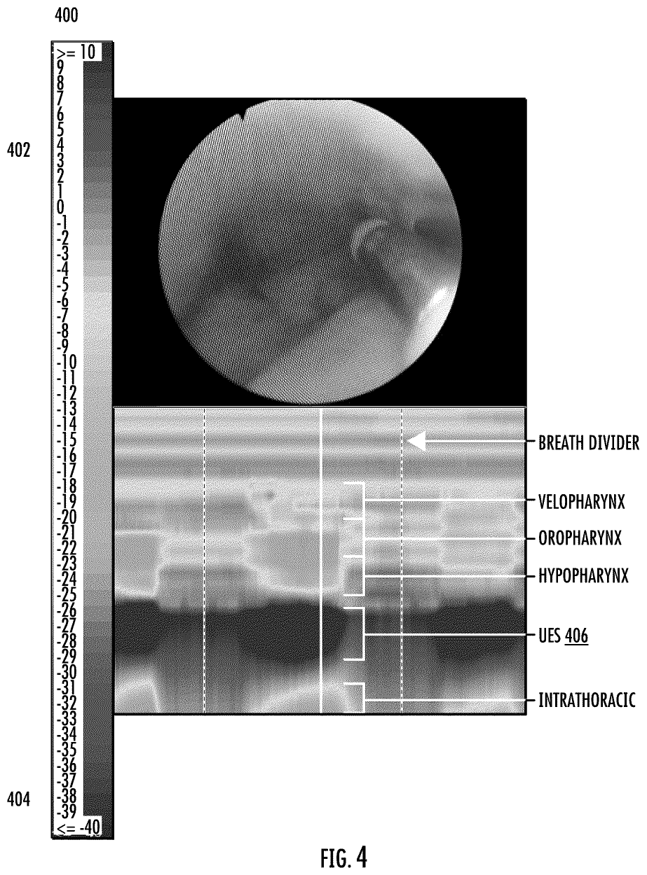

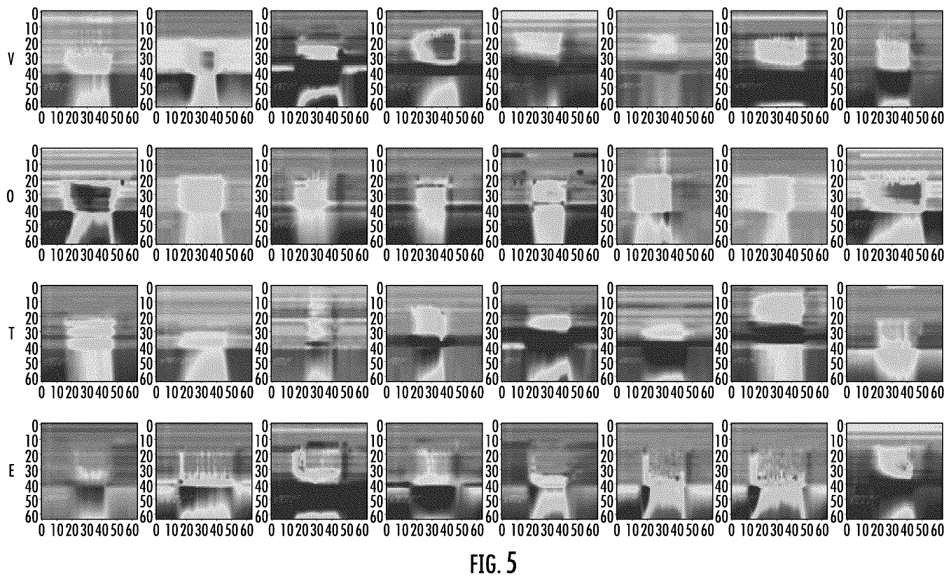

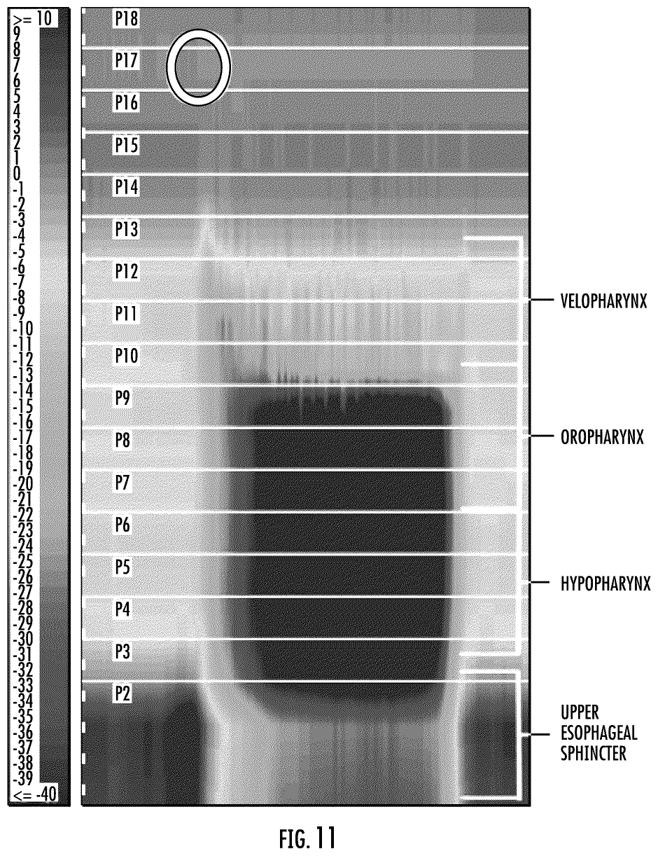

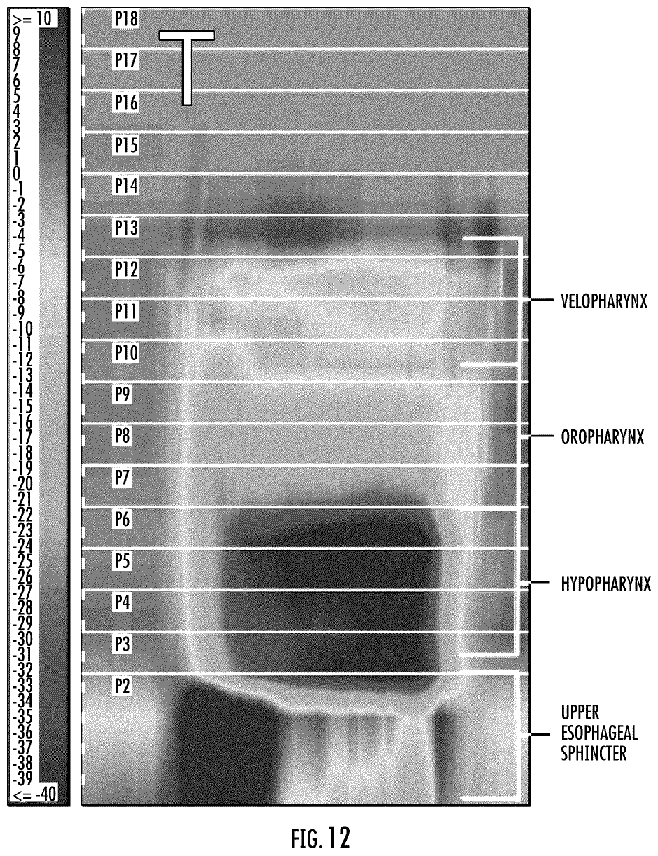

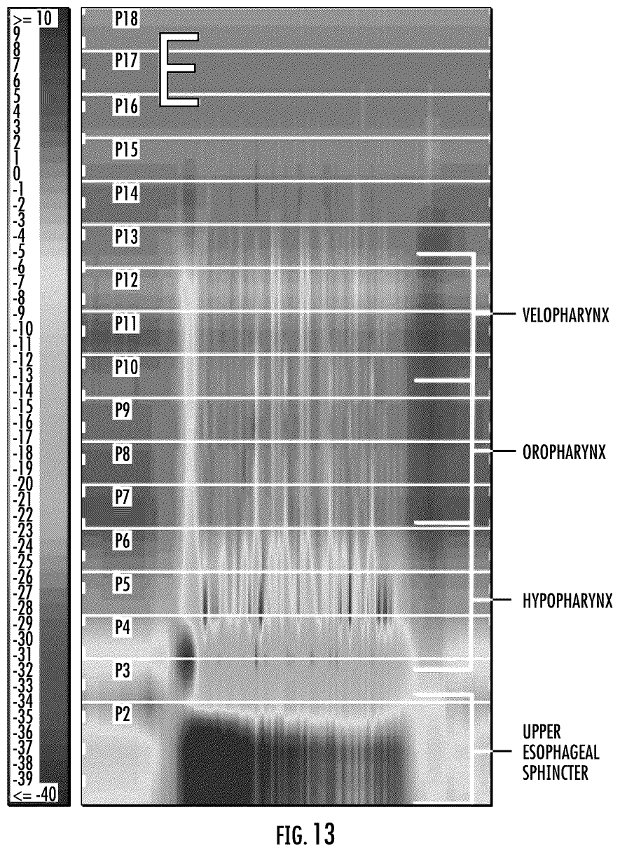

The present disclosure also contemplates generating display data for the location of pharyngeal collapse, and that such display data can be presented on a display device (for example, the output device 212 illustrated in FIG. 2). Example display data include, but are not limited to, the manometry data shown in FIG. 4 and the heatmaps shown in FIGS. 5, and 10-13. For example, non-limiting examples of the display data can include a text-based description of the location of pharyngeal collapse that can be presented on the display device. Examples of including text-based descriptions in display data are shown in FIGS. 4 and 10-13, for example. Another non-limiting example of display data is a heatmap image associated with a breath from the manometry data (see e.g., FIGS. 5 and 10-13), which the present disclosure also contemplates can be presented on a display device. It should be understood that the present disclosure contemplates that any combination of the manometry data 120, features extracted 130 and/or predictions 140 can be presented on the display device.

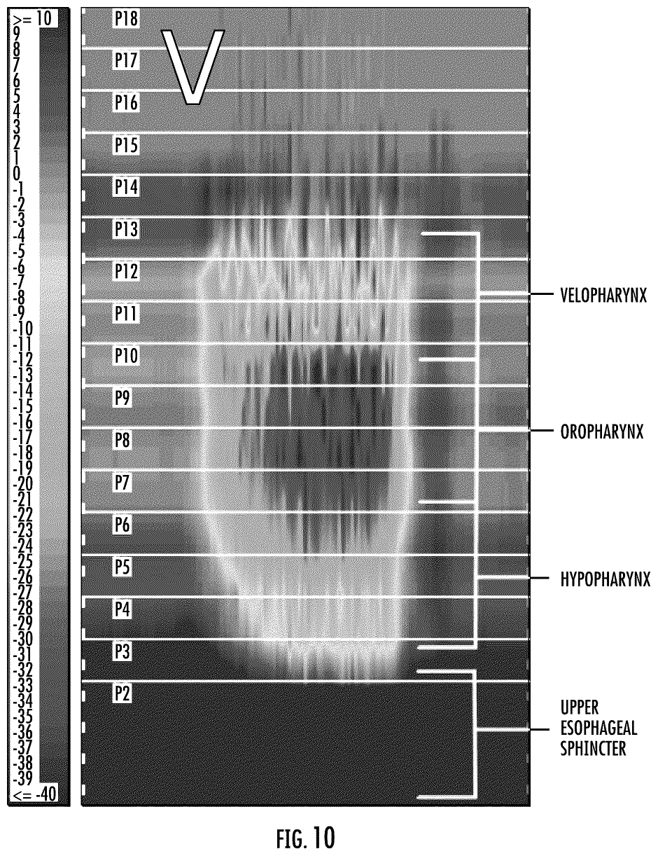

Additionally, some implementations of the present disclosure can include selecting a representative breath (or inspiration) from the manometry data and a corresponding heatmap image associated with the representative breath; and presenting the corresponding heatmap image on a display device. Examples of single-breath heatmaps are shown in FIGS. 10-13. Again, it should be understood that the display device described herein can part of the computing device illustrated in FIG. 2, for example the output device 212.

In some implementations, pharyngeal phenotyping in obstructive sleep apnea can be performed using the methods described above. Based on the pharyngeal phenotyping, the methods optionally include recommending the subject for an intervention based on the at least one of the location of pharyngeal collapse and/or the degree of pharyngeal collapse, which can be predicted by the trained machine learning model 100. It should be understood that the subject may be recommended for a plurality of interventions. Additionally, such recommendation may optionally be a classification (e.g., subject recommended or not recommended for intervention) or optionally a predicted probability of successful procedural outcome. The method can also include performing the intervention on the subject. The present disclosure contemplates that the intervention can be a surgical, medical, or device-based intervention. A non-limiting example of an intervention includes implanting a medical device in the subject, the medical device being configured to deliver nerve stimulation therapy. This disclosure contemplates that neurostimulation targets may include those affecting upper airway patency including, but not limited to, the hypoglossal nerve, the ansa cervicalis nerve plexus, the pharyngeal nerve plexus, the phrenic nerve, the glossopharyngeal nerve, the vagus nerve, the trigeminal nerve, the carotid plexus, or the internal branch of the superior laryngeal nerve. Optionally, in some implementations, the implanted medical device is configured to deliver hypoglossal nerve stimulation therapy (see e.g., the Examples described below).

Additionally, it should be understood that the methods described above with reference to FIG. 1A can be implemented using a system, for example, a system including some or all of the components illustrated in FIG. 2. In one implementation of the present disclosure, the system includes at least one processor; and a memory operably coupled to the at least one processor, where the memory has computer-executable instructions that cause the at least one processor to perform any of the methods described above. This can include receiving the manometry data 120 for the subject, and extracting a plurality of features 130 from the manometry data. Again, non-limiting examples of features 130 that can be extracted include one or more of a high-level breath feature, a frequency feature, or an LNCC feature. The extracted features 130 can be input into a trained machine learning model 100. The system can be configured to receive from the trained machine learning model 100 a prediction 140. As a non-limiting example, the prediction 140 can include a location of pharyngeal collapse for the subject and/or a degree of pharyngeal collapse for the subject. In another non-limiting example, the prediction 140 can include whether the subject is a favorable or non-favorable candidate for a medical intervention such as a nerve stimulation therapy.

Referring now to FIG. 1B, an example method 150 for training a machine learning model for pharyngeal phenotyping in obstructive sleep apnea is shown. The method 150 includes receiving respective manometry data for each of a plurality of patients 152; receiving identification of a plurality of flow-limited breaths 154; receiving a respective location of pharyngeal collapse for each of the identified flow-limited breaths 156; and receiving a respective degree of pharyngeal collapse for each of the identified flow-limited breaths 158. As described herein, a plurality of heatmaps (see e.g., FIGS. 5 and 10-13) can be generated from the manometry data 152. Additionally, the heatmaps can be associated with individual inspirations (see e.g., FIGS. 10-13). As used herein, a breath may be an inspiration, an expiration, or both inspiration and expiration. In the examples described herein, the data received at step 154 is associated with flow-limited inspirations. This disclosure contemplates that the systems and methods described herein can also be used to analyze expiratory obstruction (e.g., flow-limited expirations). This disclosure contemplates heatmaps associated with flow-limited breaths can be identified (see step 154). Breaths can be identified by analyzing manometry data, for example, using heuristic algorithms to identify breath boundaries based on the maximum pressure signatures across the sensors. It should be understood that such heuristic algorithms are provided only as an example. This disclosure contemplates using any known techniques to identify separate breaths in the manometry data. This step can be accomplished manually (e.g., by user selection) or automatically (e.g., by software). Optionally, the heatmaps associated with non-flow-limited breaths can be ignored or discarded. Thereafter, a respective location of pharyngeal collapse (see step 156) and/or a respective degree of pharyngeal collapse (see step 158) can be identified for each of the heatmaps associated with flow-limited breaths. This step can be accomplished manually (e.g., by user selection) or automatically (e.g., by software). It should be understood that the respective locations and/or degrees of pharyngeal collapse are the “labels.”

The method also includes creating a training dataset 160 including the respective manometry data for each of the patients 152, the respective location of pharyngeal collapse for each of the identified flow-limited breaths 156, and the respective degree of pharyngeal collapse for each of the identified flow-limited inspiration 158. The training dataset 160 is a labeled dataset. Additionally, the present disclosure contemplates that the training dataset 160 can optionally further include respective patient data for the plurality of patients. As described herein, patient data can include, but is not limited to, anthropomorphic data, demographic data, PSG data, video data, or electronic medical record data. The training dataset 160 can be used for training 162 the machine learning model. As described herein, the machine learning model can be a supervised learning model such as a k-NN classifier, a support vector machine classifier, a logistic regression classifier, a random forest classifier, a Naïve Bayes classifier, or an ANN. This disclosure contemplates using an appropriate machine learning training algorithm for a particular model, which are well known in the art. For example, this disclosure contemplates using any algorithm that finds the maximum or minimum of the objective function for training the machine learning model. As described herein, the trained machine learning model is configured to predict at least one of a location of pharyngeal collapse for a subject and/or a degree of pharyngeal collapse for the subject. Alternatively, the trained machine learning model can be configured to predict whether the subject is a favorable or unfavorable candidate for an intervention.

As described herein, the training dataset created in step 160 can include a plurality of features associated with the respective manometry data for each of the patients. Non-limiting examples of these features include high-level breath features, frequency features, and LNCC features. Additionally, in some implementations, the present disclosure contemplates that feature selection can optionally be performed to select a set of predictive features among the plurality of features. Feature selection can be performed empirically, for example, by iteratively training and testing different versions of a machine learning model to determine which feature set results in the best performance. Each of FIGS. 10-13 is labeled with the top five machine learning classifier features. It should be understood that FIGS. 10-13 and their respective features sets are provided only as examples. This disclosure contemplates that other features and/or feature sets may be predictive. For example, Table 1 in the Examples below illustrates example extracted features that can be used with the systems and methods described herein. The features in Table 1 reference sensors of the high-resolution manometry catheter of FIG. 3. Alternatively or additionally, in some implementations, the method optionally includes augmenting the training dataset to include a plurality of synthetic manometry data samples. Data augmentation can address issues associated with small dataset size and/or unbalanced datasets.

It should be understood that machine learning models are trained to find patterns present in datasets (or data sets). The dataset can be stored in a data storage medium accessible by a computing device (e.g., computing device 200 of FIG. 2), and optionally stored in one or more databases. This disclosure contemplates that the dataset is a labeled dataset, where the data is tagged with “labels,” which may include, but are not limited to, respective locations and/or degrees of pharyngeal collapse for patients (i.e., when training the model to predict location and/or degree of collapse) or respective outcome for patients (i.e., when training the model to predict whether a subject is a favorable or non-favorable candidate for an intervention). The labels associate the machine learning model input data (e.g., manometry data 120 and/or features 130 shown in FIG. 1A) with the machine learning model target (e.g., prediction 140 shown in FIG. 1A). Again, the predictions can include, but are not limited to, a location of pharyngeal collapse for the subject, a degree of pharyngeal collapse for the subject, or whether the subject is a favorable or non-favorable candidate for an intervention. It should be understood that the targets described herein are provided only as examples. This disclosure contemplates that targets can include other than those described herein.

It should be appreciated that the logical operations described herein with respect to the various figures may be implemented (1) as a sequence of computer implemented acts or program modules (i.e., software) running on a computing device (e.g., the computing device described in FIG. 2), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 2, an example computing device 200 upon which the methods described herein may be implemented is illustrated. It should be understood that the example computing device 200 is only one example of a suitable computing environment upon which the methods described herein may be implemented. Optionally, the computing device 200 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 200 typically includes at least one processing unit 206 and system memory 204. Depending on the exact configuration and type of computing device, system memory 204 may be volatile (such as random access memory (RAM)), non-volatile (such as read-only memory (ROM), flash memory, etc.), or some combination of the two. This most basic configuration is illustrated in FIG. 2 by dashed line 202. The processing unit 206 may be a standard programmable processor that performs arithmetic and logic operations necessary for operation of the computing device 200. The computing device 200 may also include a bus or other communication mechanism for communicating information among various components of the computing device 200.

Computing device 200 may have additional features/functionality. For example, computing device 200 may include additional storage such as removable storage 208 and non-removable storage 210 including, but not limited to, magnetic or optical disks or tapes. Computing device 200 may also contain network connection(s) 216 that allow the device to communicate with other devices. Computing device 200 may also have input device(s) 214 such as a keyboard, mouse, touch screen, etc. Output device(s) 212 such as a display, speakers, printer, etc. may also be included. The additional devices may be connected to the bus in order to facilitate communication of data among the components of the computing device 200. All these devices are well known in the art and need not be discussed at length here.

The processing unit 206 may be configured to execute program code encoded in tangible, computer-readable media. Tangible, computer-readable media refers to any media that is capable of providing data that causes the computing device 200 (i.e., a machine) to operate in a particular fashion. Various computer-readable media may be utilized to provide instructions to the processing unit 206 for execution. Example tangible, computer-readable media may include, but is not limited to, volatile media, non-volatile media, removable media and non-removable media implemented in any method or technology for storage of information such as computer readable instructions, data structures, program modules or other data. System memory 204, removable storage 208, and non-removable storage 210 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 206 may execute program code stored in the system memory 204. For example, the bus may carry data to the system memory 204, from which the processing unit 206 receives and executes instructions. The data received by the system memory 204 may optionally be stored on the removable storage 208 or the non-removable storage 210 before or after execution by the processing unit 206.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

In another example implementation, a system for identifying a location or locations of pharyngeal collapse. The system includes at least one processor; and a memory operably coupled to the processor, the memory having computer-executable instructions stored thereon. This disclosure contemplates that the processor and memory are the basic configuration is illustrated in FIG. 2 by dashed line 202. The processor is configured to receive manometry data for a subject. A plurality of pressure sensors can be used to measure the manometry data. Optionally, the pressure sensors can be part of a manometry instrument such as a pharyngeal and esophageal manometry instrument. Example pharyngeal and esophageal manometry instruments are described herein. Additionally, the pressure sensors (and/or manometry instrument) can be operably coupled to the processor and memory, for example, using one or more communication links. This disclosure contemplates the communication links are any suitable communication link. For example, a communication link may be implemented by any medium that facilitates data exchange including, but not limited to, wired, wireless and optical links.

The processor is further configured to analyze the manometry data to track a nadir position over time in the subject's airway; and identify a location of pharyngeal collapse for the subject using the nadir position over time in the subject's airway. For example, in some implementations, the processor is configured to (i) identify, in the manometry data, a percentage (e.g., top 10%) of low-pressure measurements in a breath or units of time in the breath, (ii) compute an average of the percentage (e.g., top 10%) of low-pressure measurements to create a nadir point, (ii) track the position of the nadir point over the duration of the breath, and (iv) analyze changes in the position of the nadir point over the duration of the breath to identify one or more locations of pharyngeal collapse. This disclosure contemplates analyzing changes in the position of the nadir point over the duration of the breath using statistical or machine learning methods. It should be understood that the technique for identifying locations of pharyngeal collapse described above are provided only as an example. This disclosure contemplates using other techniques. In some implementations, the system performs the analysis in real time. In other implementations, the system performs the analysis off line (i.e., post data collection). Alternatively or additionally, in some implementations, the system analyzes raw manometry data to track the nadir position over time in the subject's airway. In other implementations, the processor is further configured to extract a plurality of features from the manometry data, and the system analyzes the extracted features to track the nadir position over time in the subject's airway.

### Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Described herein is an investigation of high-resolution pharyngeal manometry (HRM) for upper airway phenotyping in OSA, including a software system that reliably predicts pharyngeal sites of collapse based solely on manometric recordings. The findings suggest that HRM may enable objective and dynamic mapping of the pharynx without a high labor cost for physician analysis, opening new pathways towards reliable and reproducible assessment of this complex anatomy in sleep.

As described below, 40 participants underwent simultaneous HRM and DISE. A machine learning algorithm was constructed to estimate pharyngeal level-specific severity of collapse, as determined by an expert DISE reviewer. The primary outcome metrics for each level were model accuracy and F1-score, which balanced model precision against recall.

During model training, the average F1-score across all categories was 0.86, with an average weighted accuracy of 0.91. Using a holdout test set of 9 participants, a k-nearest neighbor model trained on 31 participants attained an average F1-score of 0.96 and an average accuracy of 0.97. The F1-score for prediction of complete concentric palatal collapse was 0.86.

The findings described below suggest that HRM may enable objective and dynamic mapping of the pharynx, opening new pathways towards reliable and reproducible assessment of this complex anatomy in sleep.

### METHODS

### Study Design

Participants were recruited from a group of patients with OSA scheduled to undergo DISE as part of their routine clinical management plan from December 2017 to April 2019. Participants were excluded if they had any history of pharyngeal surgery aside from adenotonsillectomy, or if severe nasal airway obstruction was present that would preclude simultaneous passage of a manometry catheter and a flexible nasopharyngoscope. Demographic data including age, gender, and apnea-hypopnea index were collected for each participant.

### Experimental Procedures

### Manometry Catheter Placement

A custom-designed HRM catheter was placed in the pharynx prior to sedation. The 6-French catheter was comprised of 21 solid-state sensors spaced at 8 mm intervals, except for a 6 cm gap between the first and second most distal sensors to aid anchoring it in the esophagus (FIG. 3). The manometry catheter sensors were calibrated to atmospheric pressure after soaking the catheter in distilled water. Participants were seated in the upright position and administered intranasal oxymetazoline and 2% lidocaine jelly bilaterally before the catheter was advanced along the nasal floor.

When mild resistance to advancement at the posterior pharyngeal wall was encountered, participants were asked to maximally extend the neck to ease passage of the catheter into the oropharynx. After returning the head to the neutral position, participants then completed 1-2 dry swallows during catheter advancement to help direct it into the esophagus. Catheter position was adjusted until the distal sensor was seated in the esophagus and the adjacent group of 2-3 sensors spanned the upper esophageal sphincter (UES). The catheter was then taped to the nostril, connected to a recording system (Solar GI HRM, Laborie/Medical Measurement Systems BV, The Netherlands) and the participant was laid supine. Participants were asked to report a catheter discomfort score on a 10-point visual analog scale. Video output from the operating room endoscopy tower was directly input to the HRM recording system for synchronous recording. Each sensor recorded local pressure values in mmHg at 100 Hz.

### Drug-Induced Sleep Endoscopy

Patients were sedated for the clinical DISE exam and research experiments using standard methods.²⁹ Intravenous propofol was manually titrated throughout to maintain a bispectral index score between 50 and 70.³⁰ After stable flow-limited breathing was attained, a flexible fiberoptic nasopharyngoscope was inserted transnasally to the level of the velopharynx contralateral to the HRM catheter. Simultaneous HRM and endoscopy recordings were collected for approximately 10 minutes. Overall pharyngeal collapse patterns were documented with the VOTE (velum, oropharynx, tongue base, epiglottis) classification system.¹⁵

### Experimental Protocol

### Data Preprocessing

A custom, browser-based software interface was created for visualizing and scoring individual breath heatmaps generated from HRM data that were synchronized with high-resolution endoscopy video (illustrated in FIGS. 4 and 5). Within each heatmap, time is represented on the x-axis, with the anatomic position of each sensor represented on the y-axis. Instantaneous pressure values were coded by color, with atmospheric pressure at the warm (red) end of the spectrum transitioning to cooler (blue) colors at the most negative recorded values. Positive pressure values of the UES were represented by violet colors. Breaths were automatically parsed by the system from the most distal, intrathoracic manometry sensor by identifying local cyclic pressure maxima, which represented end-expiration values occurring between the more negative values of inspiratory effort. A single expert scorer (D.K.) observed all combined data recordings (endoscopy with manometry heatmaps) and electronically documented whether each inspiration was flow-limited. Inspirations without flow limitation and data artifacts such as swallowing or coughing were documented as non-diagnostic and excluded from further analyses. A VOTE classification score was subsequently recorded for each included breath for comparison to participant-level VOTE scoring. In the event of poor endoscopy visualization, the scorer estimated the individual breath collapse pattern based on the available endoscopy and HRM data.

### Data Analysis

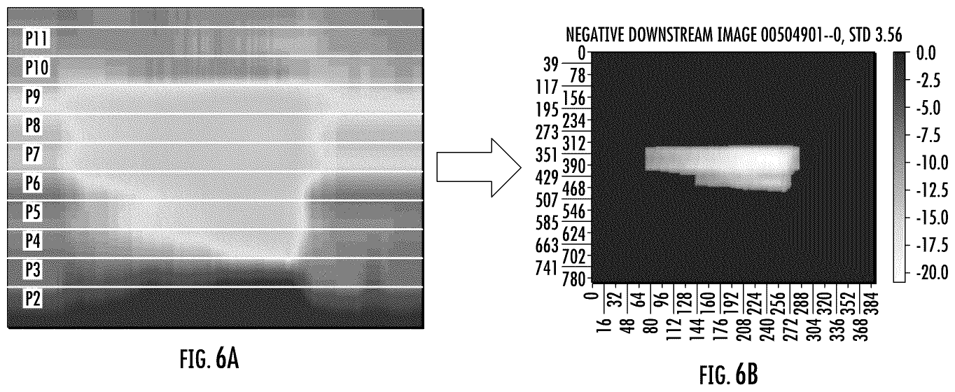

Described in this Example is a machine learning model that estimated the degree of collapse at each pharyngeal level with the VOTE classification using 92 features based on descriptions of frequent visual HRM patterns observed by the expert scorer across multiple participants, including largest negative connected component (LNCC) features. LNCCs represented the dominant shape of the negative pressure envelope within each inspiration across multiple sensors (see FIGS. 6A and 6B).

Analyses were structured to address the primary hypothesis that HRM collapse patterns could be used to accurately predict the VOTE classification score provided by an expert DISE reviewer. To address this hypothesis, a machine learning model was developed that estimated the level-specific degree of pharyngeal collapse with the VOTE classification using three classes of features: (i) high-level breath features, (ii) the frequency of per-sensor manometry value changes, and (iii) the largest negative connected component (LNCC), calculated from each manometry heatmap.

High-level breath features included simple statistics such as the average and standard deviation of manometry values across all sensors ranges of sensors for different structures of interest (e.g., sensors 10-13 for V, 7-11 for 0, 5-9 for T and 4-7 for E).

Frequency features measured the minimum, maximum, median, and standard deviation of the rate of change of manometry values per-sensor over the course of a breath, which detected rapid pressure variations caused by dynamic pharyngeal collapse.

LNCCs represented the dominant shape of the negative pressure envelope within each inspiration across multiple sensors. The shape and structure are used by machine learning models to generalize HRM patterns across patients (FIGS. 5 and 6A-6B). A LNCC was generated for each valid HRM heatmap image, from which a range of features were extracted. Extracted features included basic elements such as the width and height of an LNCC, as well as more complicated features such as the extent to which an LNCC deviates from a rectangle, specifically with respect to truncation in each corner.

In total, 92 features were defined for determining the location and degree of collapse from HRM heatmaps that were generated based on descriptions of frequent visual HRM patterns observed by the expert scorer across multiple participants (see Table 1 below). The average, median, minimum, and maximum of each feature was extracted from each breath to construct a 368-element vector for each patient.

A high number of model features can result in poor machine learning prediction if the underlying training data set is not sufficiently large because the algorithm is not able to adequately differentiate features that are important from features that induce noise. To combat this limitation, a common approach is to apply a feature selection algorithm that iteratively tests feature subsets to identify ones with the highest predictive value. Because of the small sample size, a feature selection algorithm was used during the training phase to select the best level-specific collapse pattern features (with a limit of at most four features). For example, for partial velopharyngeal collapse, sensor 5 standard deviation and sensor 7 maximum frequency features were used, while for complete velopharyngeal collapse, the bottom left corner LNCC truncation was included along with the partial velopharnygeal collapse features. Another common method for combating limited data size is data augmentation, achieved here by copying each patient's vector and adding a small amount of random noise to each parameter. This approach allowed the recorded patient data set to expand to 4,000 vectors for analysis.

Multiple supervised machine learning models were deployed to predict each participant's level-specific VOTE score, including K-nearest neighbor, support vector machine, logistic regression, random forest, and Naive Bayes. Each level-specific classification was treated as an independent binary prediction: the predictions of 0, 1 and 2 were each trained and evaluated separately for each level.

Evaluation proceeded in two phases. First, 31 patients were used to train and test classifiers using 10-fold cross validation (90% train, 10% test). Second, using the features selected during the first phase, a single model was trained with 31 patients and tested on a holdout of 9 patients. For each phase, the accuracy and F1-score were calculated for each level-specific classification. F1-scores were averaged across all level-specific classifications with an unweighted average and weighted average by level classification frequency due to class imbalances among the scored patterns. Accuracy represents a model's ability to correctly classify a label but, if the ratio of classes is highly skewed, accuracy results may be misleading. For example, if one label occurs in the data set 90% of the time, then a very simple algorithm could always pick the majority class, thus easily attaining 90% accuracy, even if the minority class is always incorrectly predicted. In contrast, the F1-score was included as an additional outcome metric, which is calculated as the harmonic mean of precision (the ability to correctly identify true positive cases of a collapse pattern) and recall (the ability to find all true positive cases of a collapse pattern in a data set), giving equal weight to both recall and precision. Intuitively, precision and recall are competing metrics: attaining perfect recall can reduce precision and vice versa. The F1-score was selected as an outcome metric as accuracy can misrepresent results if the ratio of true positives to true negatives is highly skewed. The F1-score instead balances precision (the accuracy of true positive identification) against recall (the ability to identify all true positive cases). In this Example, F1-scores were averaged across all level-specific classifications with an unweighted average and weighted average by level classification frequency due to class imbalances among the scored patterns.

Student's t-test and Fisher's Exact tests were used for evaluation of differences in demographic and polysomnographic variables of included and excluded patients, using a p-value of less than 0.05 for significance.

### Results

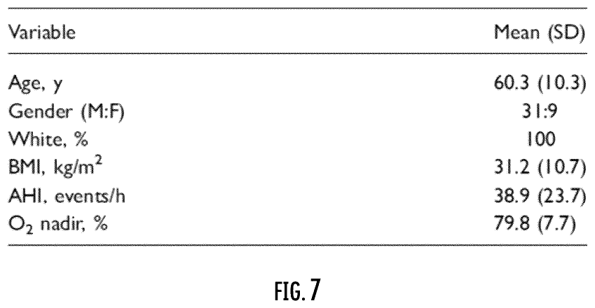

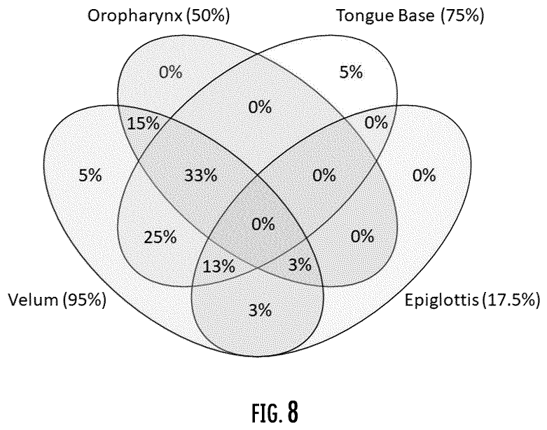

Sixty participants were consented for the study. Three participants withdrew consent before initiation of study procedures, and two participants declined to complete HRM catheter placement due to procedural discomfort, primarily due to difficulty transitioning passing the catheter from the nasal cavity into the oropharynx where the metal catheter tip would occasionally seat in uneven adenoid tissue or the fossa of Rosenmuller. All five participants were excluded from further analysis. Fifty-five participants completed all study procedures, reporting a mean catheter discomfort score of 1.5±1.3 (mean±SD) on a 10-point visual analog scale. During data analysis, 15 participants were excluded from scoring procedures due to technical errors in the collection of HRM data (primary due to technical issues resulting in incomplete endoscopy video capture in the earliest recruited participants), leaving 40 participants for analysis (FIG. 7). Included participants were primarily elderly and male, with severe OSA. There were no significant demographic differences between included and excluded participants (p>0.05). The distribution of overall VOTE collapse patterns for included participants is depicted in FIG. 8.

### Machine Learning/Data Mining Results

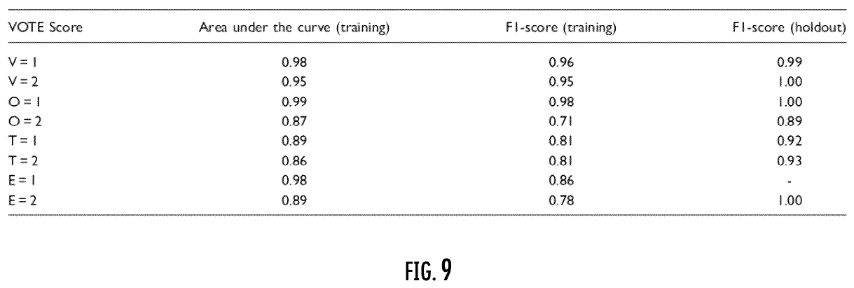

Individual breaths were found to have low predictive power for estimating either breath- or patient-level VOTE scores (data not shown). When estimating patient-level VOTE scores, the best results were achieved by a K-nearest neighbor model (KNN). For the training phase, the model predicted the degree of collapse for a given pharyngeal structure using only HRM data, with receiver operating characteristic area-under-the-curve values ranging from 0.89 to 0.98 and F1-scores ranging from 0.71 to 0.98 (FIG. 9). The F1-score for prediction of CCC was 0.89. The average weighted and unweighted F1-score across all categories was 0.86, with an average weighted and unweighted accuracy of 0.91 and 0.90, respectively. On the holdout set, the machine learning model attained an average F1-score of 0.96 and an average accuracy of 0.97. Weighting did not affect the results. The F1-score for prediction of CCC was 0.86.

A variety of machine learning features exhibited predictive value depending on the level and degree of collapse (FIGS. 10-13). In general, measurements based on frequency data from a few sensors provided the greatest predictive value for the different classifiers, although the relevant sensor or sensors varied depending on the classifier of interest (see Tables 3-11 below). At least one LNCC feature was in the top 5 most important features for each classifier except for partial tongue base collapse.

### Discussion

This study demonstrated that automated HRM processing by a machine learning algorithm can accurately summarize the sites and degree pharyngeal collapse using the VOTE classification, a system widely understood by sleep surgeons. The algorithm achieved excellent classification ability during training with area-under-the-curve values ranging from 0.89 to 0.98. When tested against a previously unseen holdout set, the HRM algorithm achieved F1-scores for pharyngeal structure and degree of collapse ranging from 0.89 to 1.0, indicating excellent to near-perfect agreement with a human DISE scorer. These findings suggest that HRM may be a useful tool for assessing the complex and dynamic collapse patterns of the pharynx in sedated or natural sleep, potentially opening new pathways toward objective assessment of the upper airway before and after sleep surgery interventions.

Individual sensor frequency information was important to several of the KNN classifiers, suggesting that this type of data has high predictive value for ascertaining the structure and degree of collapse within the pharyngeal column. Nevertheless, relevant sensors ranged from positions just above the UES to the level of the velopharynx, implying that the high density of sensors on the HRM catheter was important for generating useful classifier information. Interestingly, important sensors were not always co-located to the site of collapse. For instance, sensor 3 yielded important classifier information for partial velopharyngeal collapse, even though it was located just above the UES. Results suggest that there is a complex interplay between different pharyngeal structures during dynamic collapse.

There are several key differences between this study and prior evaluations of upper airway manometry,^{26-28, 31-33} primarily its ability to map complex pressure patters to the VOTE classification, a system readily understood by sleep surgeons. From a technical perspective, the HRM system described herein utilized closely spaced sensors that enabled high resolution mapping of the entire pharynx, even if the relative catheter position shifted. This close sensor spacing enabled the HRM system to achieve excellent discrimination and agreement with the DISE reviewer even during classification of oro- and hypopharyngeal collapse patterns. For example, the system described herein utilized 21 sensors spaced less than a centimeter apart, which permitted high-resolution mapping of the upper airway at multiple levels, with differentiation of oro- and hypopharyngeal structural collapse. The close spacing of the sensors with spanning of the upper esophageal sphincter (UES) makes our analysis less sensitive to relative catheter position within the upper airway as distance from the UES can always be easily ascertained in manual and automated analyses. Prior manometry studies also did not differentiate between the lateral pharyngeal walls, tongue base, and epiglottis during inspiratory collapse, although these structures have prognostic value for surgical planning: prior large assessments of DISE outcomes suggest that lateral wall and epiglottic collapse can substantially affect pharyngeal and hypoglossal nerve stimulator surgical outcomes.^{34, 35} The system disclosed herein was also constructed from solid-state sensors on a 6-French catheter, indicating it can be used during natural sleep, as prior investigations of manometry for surgical phenotyping in natural sleep used a 5-sensor catheter of larger diameter.^{27, 28}

Multiple machine learning models were evaluated for accuracy and F1-scores including KNN, support vector machines, logistic regression, random forest, and Naive Bayes. Empirical training and validation results demonstrated the KNN classifier performed best in the experimental setup, likely due to our limited data set. The optimal decision boundaries for HRM predictions are likely not linear, and variations among patients even within a single class can make predictions within small data sets difficult. More data-intensive models, such as support vector machines, struggle to construct accurate decision boundaries with smaller data sets. The KNN classifier likely performed well as it functions by clustering similar patients, which can work well even for small data sets. As more data are collected, more advanced and data-intensive classifiers are expected to perform better.

Results show high to perfect inter-rater agreement for degree of airway collapse between a single expert DISE reviewer and an automated machine learning algorithm evaluating HRM recordings, suggesting that HRM may have value as a diagnostic tool for dynamic upper airway collapse, especially if tolerable in natural sleep. DISE is expensive to complete in the operating room setting and requires technical expertise to balance sedation level against upper airway collapse and to accurately interpret the results.^{12, 13, 13, 14, 17, 18, 20-22, 36} The subjective nature of the exam introduces variability in inter- and intra-rater agreement when visually grading the level of anatomic collapse.³⁶⁻³⁹ Agreement between scorers declines significantly when assessing finer degrees of detail, such as the degree and pattern of collapse for a given structure, and there is a known learning curve for accurate interpretation.^{36, 38, 39} There is additionally significant disagreement between natural and drug-induced sleep endoscopy, especially at the level of the tongue base.²³ Objective pressure recordings of the upper airway are not subject to scoring variability if analysis rules are algorithmically codified. Further work can be conducted to assess whether such a catheter system is tolerable during natural sleep, and whether a scoring system's accuracy remains high across multiple expert scorers.

Airway collapse patterns have been observed to change during the course of a DISE examination, and it would be desirable for this HRM algorithm to report the distribution of all observed collapse patterns for a single patient. This task was attempted during exploratory analyses by scoring the VOTE pattern of each inspiration with observed collapse on endoscopy but discovered that LNCCs from single breaths were poorly predictive of their respective VOTE scores (data not shown). Further investigation revealed that while all scored breaths displayed visual evidence of reduced airway caliber, only 10% of them generated significant negative pressure gradients discernable as a distinct LNCC against the background of normal mildly negative inspiratory airflow pressures. These findings would suggest that either the manometry catheter was insensitive to negative pressure changes within the upper airway, or that the human reviewer was overly sensitive to airway collapse, scoring many breaths with positive collapse findings despite an absence of significant negative pressure gradients expected during flow-limited inspiration. Per the manufacturer's documentation (Unisensor AG, Switzerland), the catheter's sensors are accurate to within ±2% of the true value from −50 to 300 mmHg, suggesting the hardware was sufficiently sensitive and accurate for this application. Accurate manometry hardware implies that our expert scorer was overly sensitive to airway collapse, although he has participated in multiple large reviews of DISE where consensus agreement among expert scorers was on par with agreement rates reported elsewhere in the literature.^{34, 35} Another viable explanation is that endoscopic changes in pharyngeal cross-sectional area may not always correlate well with the site and degree of inspiratory flow limitation. Prior work reports that inspiratory airflow is independently related to cross-sectional area as well as driving pressure across the velopharynx, suggesting that, at a given cross-sectional area, airflow limitation may be present at one inspiratory driving pressure and absent at another. Prior work also suggests that the structure observed to collapse may also not represent the primary site of flow limitation,⁴⁰ and may not even be present in the endoscopy field as DISE can be inherently limited by the position of the endoscope: too proximal, and the primary site of collapse may be obscured by a partially collapsed proximal structure; too distal, and the primary site of flow limitation may rest behind the viewer, more proximal in the airway. HRM, however, maintains a global view of the upper airway due to the presence of multiple simultaneously recording sensors, and generation of negative pressure gradients between sensors only occurs when site-specific flow limitation exists.

This example describes a machine learning algorithm that correlates HRM data with the VOTE classification, a visual phenotyping scale that is familiar to sleep surgeons. Nevertheless, aspects of the HRM data suggest it may have several advantages to DISE. Foremost are the objective nature of the collected data, as well as the ability to visualize and evaluate pressure changes in different areas of the pharynx simultaneously (i.e., global evaluation of the pharynx). Simultaneous data capture from multiple sites creates the potential for interesting new analyses of airway change through the respiratory cycle. For instance, some recorded breaths suggest that proximal sites of flow resistance and decreasing pressure early in the inspiratory phase may trigger collapse of susceptible downstream structures raising the question as to whether the patient would benefit more from management of the proximal site of collapse earlier in the inspiration or the later, distal site of collapse. Pre- and post-management HRM testing may help to elucidate the answers to these questions and others.

This study only explored the agreement for pharyngeal site and degree of collapse between DISE and HRM and did not evaluate DISE findings in more granular detail. Nevertheless, the analysis for presence of CCC suggests that HRM systems have the potential to identify meaningful patterns for the most detailed levels of DISE evaluation. Based on the analyses, this disclosure contemplates that more data can be collected for machine learning algorithms to extract meaningful associations. Further data collection is also anticipated to provide enough data for application of more complex machine learning models, such as deep neural networks. Additionally, agreement between HRM and DISE was assessed for only one expert reviewer in this study. Further studies can be conducted to assess interrater agreement between a machine learning algorithm and multiple expert DISE reviewers. Additionally, substantial class imbalances existed in the data, with a high proportion of patients exhibiting palatal and tongue base collapse. Nevertheless, the distribution of collapse patterns is similar to other large case series in the literature,⁴¹ and the system described herein performed well even against structures that collapsed less frequently, such as the epiglottis and oropharyngeal lateral walls. Additionally, further studies can be conducted to better elucidate the discrepancies observed between the majority of HRM and DISE breaths, where observed dynamic airway collapse did not translate to negative pharyngeal pressure gradients. Regardless, exclusion of this significant percentage of breaths did not appear to substantially impact the algorithm's patient-level agreement with the expert scorer for the degree of structural collapse.

This study described above suggests that HRM findings associate well with DISE collapse patterns in the upper airway that are well understood by surgeons. It may have utility as a diagnostic tool for objective, phenotyping of complex, dynamic collapse patterns of the upper airway during DISE and possibly even natural sleep.

Table 1 below illustrates 92 features defined for determining the location and degree of collapse from HRM heatmaps. These features were generated based on descriptions of frequent visual HRM patterns observed by the expert scorer across multiple participants. Features were generated from one of three classes: (i) high-level breath features, (ii) the frequency of per-sensor manometry value changes, and (iii) the largest negative connected component (LNCC), calculated from each manometry heatmap.

TABLE 1

| Index | Type | Feature | Definition |
|---|---|---|---|
| 1 | Breath | avg_all_value | The average manometry value of |
| | Structure | | the breath |
| 2 | Breath | avg_negative_value | The average negative manometry |
| | Structure | | value of the breath |
| 3 | Breath | avg_breath_V | The average manometry value of |
| | Structure | | the breath between sensor 10-13 |
| 4 | Breath | avg_breath_O | The average manometry value of |
| | Structure | | the breath between sensor 7-11 |
| 5 | Breath | avg_breath_T | The average manometry value of |
| | Structure | | the breath between sensor 5-9 |
| 6 | Breath | avg_breath_E | The average manometry value of |
| | Structure | | the breath between sensor 4-7 |
| 7 | Breath | avg_neg_V | The average negative manometry |
| | Structure | | value of the breath between sensor |
| | | | 10-13 |
| 8 | Breath | avg_neg_O | The average negative manometry |
| | Structure | | value of the breath between sensor |
| | | | 7-11 |
| 9 | Breath | avg_neg_T | The average negative manometry |
| | Structure | | value of the breath between sensor |
| | | | 5-9 |
| 10 | Breath | avg_neg_E | The average negative manometry |
| | Structure | | value of the breath between sensor |
| | | | 4-7 |
| 11 | Breath | std_all_value | The standard deviation of the |
| | Structure | | manometry value of the breath |
| 12 | Breath | std_negative_value | The standard deviation of the |
| | Structure | | negative manometry value of the |
| | | | breath |
| 13 | Frequency | sensor_{3~14}_max_freq | The maximum frequency at sensor |
| | | | 3-14 |
| 14 | Frequency | sensor_{3~14}_mean_freq | The average frequency at sensor 3- |
| | | | 14 |
| 15 | Frequency | sensor_{3~14}_median_freq | The median frequency at sensor 3- |
| | | | 14 |
| 16 | Frequency | sensor_{3~14}_std_freq | The standard deviation of the |
| | | | frequency at sensor 3-14 |
| 17 | LNCC | height | The height of the LNCC (number of |
| | Structure | | pixels) |
| 18 | LNCC | width | The time length of the LNCC |
| | Structure | | (number of pixels) |
| 19 | LNCC | height_div_width | The height of the LNCC divided by |
| | Structure | | the width of the LNCC |
| 20 | LNCC | length_second | The time length of the LNCC (in |
| | Structure | | seconds) |
| 21 | LNCC | rec_end_sensor | The end sensor of the LNCC |
| | Structure | | |
| 22 | LNCC | rec_start_sensor | The start sensor of the LNCC |
| | Structure | | |
| 23 | LNCC | rect_height_sensor | The height of the LNCC (number of |
| | Structure | | sensors) |
| 24 | LNCC | rect_length_second | The time length of the LNCC (in |
| | Structure | | seconds) |
| 25 | LNCC | Sensor_div_time | The height of the LNCC (the |
| | Structure | | number of sensors) divided by the |
| | | | time length of the LNCC (in |
| | | | seconds) |
| 26 | LNCC | time_centroid | Normalized the pixel centroid of |
| | Structure | | the LNCC by its duration of the |
| | | | breath |
| 27 | LNCC | left_top | Number of zero pixels at left top |
| | Structure | | corner of the rectangle boundary |
| | | | of the LNCC divided by the sum of |
| | | | zero pixels at each corner of the |
| | | | rectangle boundary of the LNCC |
| 28 | LNCC | left_bottom | Number of zero pixels at left |
| | Structure | | bottom corner of the rectangle |
| | | | boundary of the LNCC divided by |
| | | | the sum of zero pixels at each |
| | | | corner of the rectangle boundary |
| | | | of the LNCC |
| 29 | LNCC | right_top | Number of zero pixels at right top |
| | Structure | | corner of the rectangle boundary |
| | | | of the LNCC divided by the sum of |
| | | | zero pixels at each corner of the |
| | | | rectangle boundary of the LNCC |
| 30 | LNCC | right_bottom | Number of zero pixels at right |
| | Structure | | bottom corner of the rectangle |
| | | | boundary of the LNCC divided by |
| | | | the sum of zero pixels at each |
| | | | corner of the rectangle boundary |
| | | | of the LNCC |
| 31 | LNCC | bottom_up_negative_ratio | The sum of the negative values at |
| | Structure | | the bottom part of the LNCC |
| | | | divided by the sum of the negative |
| | | | values at the top part of the LNCC. |
| 32 | LNCC | left_right_negative_ratio | The sum of the negative values at |
| | Structure | | the left part of the LNCC divided by |
| | | | the sum of the negative values at |
| | | | the right part of the LNCC. |
| 33 | LNCC | temporal_negative_ratio | The sum of the negative values at |
| | Structure | | the right part of the LNCC divided |
| | | | by the sum of the negative values |
| | | | at the left part of the LNCC. |
| 34 | LNCC | up_bottom_negative_ratio | The sum of the negative values at |
| | Structure | | the top part of the LNCC divided by |
| | | | the sum of the negative values at |
| | | | the bottom part of the LNCC. |
| 35 | LNCC | left_bottom_corner_circle_rad_second | Find a circle that can be perfectly |
| | Structure | | fitted into the left bottom, |
| | | | compute the height of the |
| | | | rectangle, and normalize the height |
| | | | by dividing it with the height of a |
| | | | sensor. |
| 36 | LNCC | left_bottom_corner_circle_rad_sensor | Find a circle that can be perfectly |
| | Structure | | fitted into the left bottom, |
| | | | compute the length of its diagonal, |
| | | | and normalize the length into |
| | | | second. |
| 37 | LNCC | left_bottom_corner_rect_height_sensor | Find a rectangle that can be |
| | Structure | | perfectly fitted into the left |
| | | | bottom, compute the height of the |
| | | | rectangle, and normalize the height |
| | | | by dividing it with the height of a |
| | | | sensor. |
| 38 | LNCC | left_bottom_corner_rect_length_second | Find a rectangle that can be |
| | Structure | | perfectly fitted into the left |
| | | | bottom, compute the length of its |
| | | | diagonal, and normalize the length |
| | | | into second. |
| 39 | LNCC | right_bottom_corner_circle_rad_second | Find a circle that can be perfectly |
| | Structure | | fitted into the right bottom, |
| | | | compute the radius, and normalize |
| | | | the radius into second. |
| 40 | LNCC | right_bottom_corner_circle_rad_sensor | Find a circle that can be perfectly |
| | Structure | | fitted into the right bottom, |
| | | | compute the radius, and normalize |
| | | | the radius by dividing it with the |
| | | | height of a sensor. |
| 41 | LNCC | negative_centroid_sensor | The pixel centroid of the negative |
| | Structure | | value of the LNCC normalized to |
| | | | the number of sensors |
| 42 | LNCC | negative_geometric_distance | The distance of the centroid of the |
| | Structure | | negative value of the LNCC and the |
| | | | centroid of the LNCC |
| 43 | LNCC | negative_percentage_rectangle | The percentage of negative values |
| | Structure | | within the rectangle boundary of |
| | | | the LNCC |
| 44 | LNCC | negative_rect_time_percentage | The length of the LNCC (in seconds) |
| | Structure | | divided by the length of the breath |
| | | | (in seconds) |
| 45 | LNCC | square_ratio | The area of the LNCC divided by |
| | Structure | | the area of the rectangle boundary |
| | | | of the LNCC |
| 46 | LNCC | extreme_negative_centroid_sensor | The distance of centroid of the |
| | Structure | | negative value of the LNCC and the |
| | | | position of the extreme negative |
| | | | value of the LNCC |
| 47 | LNCC | extreme_negative_geometric_distance | The distance of centroid of the |
| | Structure | | LNCC and the position of the |
| | | | extreme negative value of the LNCC |
| 48 | LNCC | geometric_centroid_sensor | The pixel centroid of the LNCC |
| | Structure | | normalized to the number of |
| | | | sensors |

The following is a confusion matrix analysis for level of degree or collapse. These analyses are based on the best data mining rules, KNN, with data augmentation.

TABLE 2
Breakdown of level and degree of collapse
represented within the dataset

| Pattern | 0 | 1 | 2 |
|---|---|---|---|
| V | 2 | 6 | 23 |
| O | 16 | 4 | 11 |
| T | 7 | 9 | 15 |
| E | 25 | 2 | 4 |

V=1
AUC=0.98
F1=0.96
Best rule: [[‘sensor_5_std_freq’, ‘sensor_7_max_freq’]]

### Normalized Confusion Matrix:

| Label | V is not 1 | V is 1 |
|---|---|---|
| V is not 1 | 0.71 | 0.04 |
| V is 1 | 0.00 | 0.25 |

### Confusion Matrix:

| Label | V is not 1 | V is 1 |
|---|---|---|
| V is not 1 | 15.63 | 1.24 |
| V is 1 | 0.00 | 7.75 |

V=2
AUC=0.95
F1=0.95
[[‘left_bottom’, ‘sensor_7_max_freq’, ‘sensor_5_std_freq’]]

### Normalized Confusion Matrix:

| Label | V is not 2 | V is 2 |
|---|---|---|
| V is not 2 | 0.21 | 0.04 |
| V is 2 | 0.03 | 0.72 |

V, C=2
AUC=0.96
F1=0.89
[[‘right_bottom’, ‘sensor_11_std_freq’, ‘sensor_7_std_freq’, ‘temporal_negative_ratio’, ‘sensor_10_std_freq’]]

### Normalized Confusion Matrix:

| Label | V is not C2 | V is C2 |
|---|---|---|
| V is not C2 | 0.65 | 0.10 |
| V is C2 | 0.00 | 0.25 |

O=1
AUC=0.99
F1=0.98
[[‘negative_percentage_rectangle’, ‘right_bottom’, ‘right_top’]]

### Normalized Confusion Matrix:

| Label | O is not 1 | O is 1 |
|---|---|---|
| O is not 1 | 0.73 | 0.02 |
| O is 1 | 0.00 | 0.25 |

O=2
AUC=0.87
F1=0.71
[[‘sensor_12_median_freq’, ‘std_all_value’]]

### Normalized Confusion Matrix:

| Label | O is not 2 | O is 2 |
|---|---|---|
| O is not 2 | 0.64 | 0.11 |
| O is 2 | 0.04 | 0.21 |

T=1
AUC=0.89
F1=0.81
[[‘sensor_8_mean_freq’, ‘std_negative_value’, ‘sensor_11_median_freq’]]

### Normalized Confusion Matrix:

| Label | T is not 1 | T is 1 |
|---|---|---|
| T is not 1 | 0.70 | 0.05 |
| T is 1 | 0.03 | 0.22 |

T=2
AUC=0.86
F1=0.81
[[‘rec_start_sensor’, ‘sensor_4_median_freq’, ‘std_all_value’, ‘sensor_14_std_freq’]]

### Normalized Confusion Matrix:

| Label | T is not 2 | T is 2 |
|---|---|---|
| T is not 2 | 0.44 | 0.06 |
| T is 2 | 0.11 | 0.39 |

E=1
AUC=0.98
F1=0.86
[[‘negative_geometric_distance’, ‘sensor_6_median_freq’]]

### Normalized Confusion Matrix:

| Label | E is not 1 | E is 1 |
|---|---|---|
| E is not 1 | 0.90 | 0.04 |
| E is 1 | 0.00 | 0.06 |

E=2
AUC=0.89
F1=0.78
[[‘left_right_negative_ratio’, ‘sensor_11_max_freq’, ‘std_negative_value’]]

### Normalized Confusion Matrix:

| Label | E is not 2 | E is 2 |
|---|---|---|
| E is not 2 | 0.74 | 0.01 |
| E is 2 | 0.05 | 0.20 |

Tables 3-11 illustrates top rule analysis for level and degree of collapse.

K-nearest neighbor (KNN) model was applied to predict label in the patient level. KNN model does not have coefficients for the features, but the centers of patient groups can be analyzed based on the top rules identified by the KNN model. For example, the following table shows the centers (i.e., the average value of the feature within the patient group) of the patient groups (V is C2 and V is not C2).

Tables 3-11 show the average values of the top 5 features for predicting level and degree of collapse selected by the data mining process using the KNN model. By sorting all features by their frequencies of being selected by the data mining process, the top 5 features can be picked as the most important ones.

TABLE 3
Top Features for V = 1

| Feature | V is not 1 | V is 1 |
|---|---|---|
| rect_height_sensor | 6.63 | 4.73 |
| sensor_3_std_freq | 0.61 | 0.47 |
| right_bottom_corner_circle_rad_sensor | 0.4 | 0.25 |
| sensor_8_mean_freq | 4.3 | 2.38 |
| rec_end_sensor | 6.92 | 9.83 |

TABLE 4
Top Features for O = 1

| Feature | O is not 1 | O is 1 |
|---|---|---|
| sensor_9_std_freq | 0.76 | 0.5 |
| sensor_4_max_freq | 4.44 | 0.69 |
| sensor_9_max_freq | 5.71 | 2.71 |
| sensor_7_median_freq | 4.5 | 1.62 |
| right_top | 0.83 | 0.82 |

TABLE 5
Top Features for T = 1

| Feature | T is not 1 | Tis 1 |
|---|---|---|
| std_negative_value | 4.74 | 3.86 |
| std_all_value | 6.2 | 5.21 |
| sensor_11_std_freq | 0.9 | 1.12 |
| sensor_3_max_freq | 3.28 | 2.13 |
| sensor_9_median_freq | 3.57 | 5.27 |

TABLE 6
Top Features for E = 1

| Feature | E is not 1 | E is 1 |
|---|---|---|
| sensor_9_max_freq | 5.15 | 7.77 |
| negative_geometric_distance | 10.31 | 8.25 |
| sensor_9_std_freq | 0.69 | 1.2 |
| sensor_7_max_freq | 5.49 | 7.64 |
| sensor_7_mean_freq | 4.07 | 5.39 |

TABLE 7
Top Features for V = 2

| Feature | V is not 2 | V is 2 |
|---|---|---|
| sensor_7_max_freq | 4.53 | 6.01 |
| right_top | 0.85 | 0.82 |
| left_bottom | 0.84 | 0.83 |
| time_centroid | 0.41 | 0.45 |
| square_ratio | 0.97 | 0.96 |

TABLE 8
Top Features for O = 2

| Feature | O is not 2 | O is 2 |
|---|---|---|
| sensor_11_median_freq | 4.16 | 5.94 |
| temporal_negative_ratio | 2.31 | 1.93 |
| std_all_value | 4.77 | 7.97 |
| sensor_12_median_freq | 4.22 | 5.92 |
| sensor_12_mean_freq | 4.28 | 5.97 |

TABLE 9
Top Features for T = 2

| Feature | T is not 2 | T is 2 |
|---|---|---|
| std_all_value | 6.14 | 5.67 |
| sensor_3_max_freq | 2.45 | 3.48 |
| sensor_3_mean_freq | 1.35 | 2.28 |
| rec_start_sensor | 13.19 | 14.53 |
| sensor_13_mean_freq | 4.82 | 1.86 |

TABLE 10
Top Features for E = 2

| Feature | E is not 2 | E is 2 |
|---|---|---|
| left_right_negative_ratio | 5.79 | 1.8 |
| sensor_11_max_freq | 7.18 | 2.05 |
| sensor_11_median_freq | 5.24 | 1.74 |
| std_negative_value | 4.77 | 2.55 |
| rec_end_sensor | 6.92 | 9.83 |

TABLE 11
Top Features for V = C2

| Feature | V is not C2 | V is C2 |
|---|---|---|
| right_bottom | 0.81 | 0.58 |
| sensor_11_std_freq | 0.91 | 1.19 |
| temporal_negative_ratio | 2.21 | 2.05 |
| sensor_7_std_freq | 0.83 | 0.93 |
| negative_percentage_rectangle | 0.74 | 0.81 |

### REFERENCES

1. Veasey S C, Rosen I M. Obstructive Sleep Apnea in Adults. Solomon C G, ed. N Engl J Med. 2019; 380 (15):1442-1449. doi:10.1056/NEJMcp1816152
2. Dempsey J A, Veasey S C, Morgan B J, O'Donnell C P. Pathophysiology of sleep apnea. Physiol Rev. 2010; 90 (1):47-112. doi:10.1152/physrev.00043.2008
3. Gottlieb D J, Yenokyan G, Newman A B, et al. Prospective study of obstructive sleep apnea and incident coronary heart disease and heart failure: the sleep heart health study. Circulation. 2010; 122 (4):352-360. doi:10.1161/CIRCULATIONAHA.109.901801
4. Yaggi H K, Concato J, Kernan W N, Lichtman J H, Brass L M, Mohsenin V. Obstructive sleep apnea as a risk factor for stroke and death. New England Journal of Medicine. Published online 2005. doi:10.1056/NEJMoa043104
5. Somers V K, White D P, Amin R, et al. Sleep Apnea and Cardiovascular Disease. An American Heart Association/American College of Cardiology Foundation Scientific Statement From the American Heart Association Council for High Blood Pressure Research Professional Education Committee, Council on. Journal of the American College of Cardiology. Published online 2008. doi:10.1016/j.jacc.2008.05.002
6. Marin J M, Carrizo S J, Vicente E, Agusti A G N. Long-term cardiovascular outcomes in men with obstructive sleep apnoea-hypopnoea with or without treatment with continuous positive airway pressure: an observational study. Lancet. 2005; 365 (9464):1046-1053. doi:10.1016/S0140-6736 (05) 71141-7
7. Kushida C A, Nichols D A, Holmes T H, et al. Effects of Continuous Positive Airway Pressure on Neurocognitive Function in Obstructive Sleep Apnea Patients: The Apnea Positive Pressure Long-term Efficacy Study (APPLES). Sleep. 2012; 35 (12):1593-1602. doi:10.5665/sleep.2226
8. Rosen C L, Auckley D, Benca R, et al. A Multisite Randomized Trial of Portable Sleep Studies and Positive Airway Pressure Autotitration Versus Laboratory-Based Polysomnography for the Diagnosis and Treatment of Obstructive Sleep Apnea: The HomePAP Study. Sleep. Published online 2012. doi:10.5665/sleep.1870
9. Kent D, Stanley J, Aurora R N, et al. Referral of adults with obstructive sleep apnea for surgical consultation: an American Academy of Sleep Medicine clinical practice guideline. J Clin Sleep Med. Published online Aug. 5, 2021. doi:10.5664/jcsm.9592
10. Croft C B, Pringle M. Sleep nasendoscopy: a technique of assessment in snoring and obstructive sleep apnoea. Clin Otolaryngol Allied Sci. 1991; 16 (5):504-509. doi:10.1111/j.1365-2273.1991.tb01050.x
11. US FDA Premarket Approval for Inspire II Upper Airway Stimulator. https://www.accessdata.fda.gov/scripts/cdrh/cfdocs/cfpma/pma.cfm?id=P130008
12. Ehsan Z, Mahmoud M, Shott S R, Amin R S, Ishman S L. The effects of Anesthesia and opioids on the upper airway: A systematic review: Anesthesia and Opioids Effects on the Upper Airway. The Laryngoscope. 2016; 126 (1):270-284. doi:10.1002/lary.25399
13. Shteamer J W, Dedhia R C. Sedative choice in drug-induced sleep endoscopy: A neuropharmacology-based review: DISE Neuropharmacology Review. The Laryngoscope. 2017; 127 (1):273-279. doi:10.1002/lary.26132
14. Cho J S, Soh S, Kim E J, et al. Comparison of three sedation regimens for drug-induced sleep endoscopy. Sleep and Breathing. 2015; 19 (2):711-717. doi:10.1007/s11325-015-1127-9
15. Kezirian E J, Hohenhorst W, de Vries N. Drug-induced sleep endoscopy: the VOTE classification. Eur Arch Otorhinolaryngol. 2011; 268 (8):1233-1236. doi:10.1007/s00405-011-1633-8
16. Vicini C, De Vito A, Benazzo M, et al. The nose oropharynx hypopharynx and larynx (NOHL) classification: A new system of diagnostic standardized examination for OSAHS patients. European Archives of Oto-Rhino-Laryngology. 2012; 269 (4):1297-1300. doi:10.1007/s00405-012-1965-z
17. De Vito A, Carrasco Llatas M, Ravesloot M J, et al. European position paper on drug-induced sleep endoscopy: 2017 Update. Clin Otolaryngol. 2018; 43 (6):1541-1552. doi:10.1111/coa.13213
18. Ghazizadeh S, Moore K, Kiai K, Mendelsohn A H. Drug-Induced Sleep Endoscopy Performed in the Endoscopy Suite: A Resource Utilization Analysis. Otolaryngol Head Neck Surg. 2020; 162 (3):386-391. doi:10.1177/0194599820901516
19. Rabelo F A W, Küpper D S, Sander H H, Fernandes R M F, Valera F C P. Polysomnographic evaluation of propofol-induced sleep in patients with respiratory sleep disorders and controls. Laryngoscope. 2013; 123 (9):2300-2305. doi:10.1002/lary.23664
20. Hillman D R, Walsh J H, Maddison K J, et al. Evolution of Changes in Upper Airway Collapsibility during Slow Induction of Anesthesia with Propofol: Anesthesiology. 2009; 111 (1):63-71. doi:10.1097/ALN.0b013e3181a7ec68
21. Eastwood P R, Platt P R, Shepherd K, Maddison K, Hillman D R. Collapsibility of the upper airway at different concentrations of propofol anesthesia. Anesthesiology. 2005; 103 (3):470-477. doi:10.1097/00000542-200509000-00007
22. Golbin D, Musgrave B, Succar E, Yaremchuk K. Clinical analysis of drug-induced sleep endoscopy for the OSA patient. Laryngoscope. 2016; 126 (1):249-253. doi:10.1002/lary.25516
23. Park D, Kim J S, Heo S J. Obstruction Patterns During Drug-Induced Sleep Endoscopy vs Natural Sleep Endoscopy in Patients With Obstructive Sleep Apnea. JAMA otolaryngology—head & neck surgery. Published online Jun. 27, 2019. doi:10.1001/jamaoto.2019.1437
24. Sleep-Related Breathing Disorders in Adults: Recommendations for Syndrome Definition and Measurement Techniques in Clinical Research. Sleep. 1999; 22 (5):667-689. doi:10.1093/sleep/22.5.667
25. Carlson D A, Pandolfino J E. High-Resolution Manometry in Clinical Practice. Gastroenterol Hepatol (NY). 2015; 11 (6):374-384.
26. Remmers J E, deGroot W J, Sauerland E K, Anch A M. Pathogenesis of upper airway occlusion during sleep. Journal of Applied Physiology. 1978; 44 (6):931-938. doi:10.1152/jappl.1978.44.6.931
27. Woodson B T, Wooten M R. A Multisensor Solid-State Pressure Manometer to Identify the Level of Collapse in Obstructive Sleep Apnea. Otolaryngol Head Neck Surg. 1992; 107 (5):651-656. doi:10.1177/019459989210700507
28. Woodson B T, Wooten M R. Manometric and Endoscopic Localization of Airway Obstruction after Uvulopalatopharyngoplasty. Otolaryngol Head Neck Surg. 1994; 111 (1):38-43. doi:10.1177/019459989411100109
29. Charakorn N, Kezirian E J. Drug-Induced Sleep Endoscopy. Otolaryngologic Clinics of North America. 2016; 49 (6):1359-1372. doi:10.1016/j.otc.2016.06.002
30. Kent D T, Zealear D, Schwartz A R. Ansa Cervicalis Stimulation: A New Direction in Neurostimulation for Obstructive Sleep Apnea. Chest. Published online October 2020:S0012369220348996. doi:10.1016/j.chest.2020.10.010
31. Singh A, Al-Reefy H, Hewitt R, Kotecha B. Evaluation of ApneaGraph in the diagnosis of sleep-related breathing disorders. European Archives of Oto-Rhino-Laryngology. 2008; 265 (12):1489-1494. doi:10.1007/s00405-008-0685-x
32. Karaloğlu F, Kemaloğlu Y K, Yilmaz M, Ulukavak Çiftçi T, Çiftçi B, Bakkal F K. Comparison of full-night and ambulatory polysomnography with ApneaGraph in the subjects with obstructive sleep apnea syndrome. Eur Arch Otorhinolaryngol. 2017; 274 (1):189-195. doi:10.1007/s00405-016-4162-7
33. Chaban R, Cole P, Hoffstein V. SITE OF UPPER AIRWAY OBSTRUCTION IN PATIENTS WITH IDIOPATHIC OBSTRUCTIVE SLEEP APNEA: The Laryngoscope. 1988; 98 (6):641???647. doi:10.1288/00005537-198806000-00013
34. Green K K, Kent D T, D'Agostino M A, et al. Drug-Induced Sleep Endoscopy and Surgical Outcomes: A Multicenter Cohort Study. The Laryngoscope. 2019; 129 (3):761-770. doi:10.1002/lary.27655
35. Huyett P, Kent D T, D'Agostino M A, et al. Drug-Induced Sleep Endoscopy and Hypoglossal Nerve Stimulation Outcomes: A Multicenter Cohort Study. The Laryngoscope. Published online Jan. 14, 2021: lary.29396. doi:10.1002/lary.29396
36. Hamans E, Kotecha B T, Kezirian E J, et al. Observer Variation in Drug-Induced Sleep Endoscopy: Experienced Versus Nonexperienced Ear, Nose, and Throat Surgeons. Sleep. 2013; 36 (6):947-953. doi:10.5665/sleep.2732
37. Gillespie M B, Reddy R P, White D R, Discolo C M, Overdyk F J, Nguyen S A. A trial of drug-induced sleep endoscopy in the surgical management of sleep-disordered breathing. Laryngoscope. 2013; 123 (1):277-282. doi:i
38. Rodriguez-Bruno K, Goldberg A N, McCulloch C E, Kezirian E J. Test-retest reliability of drug-induced sleep endoscopy. Otolaryngology—Head and Neck Surgery. 2009; 140 (5):646-651. doi:10.1016/j.otohns.2009.01.012
39. Kezirian E J, White D P, Malhotra A, Ma W, McCulloch C E, Goldberg A N. Interrater reliability of drug-induced sleep endoscopy. Arch Otolaryngol Head Neck Surg. 2010; 136 (4):393-397. doi:10.1001/archoto.2010.26
40. Garcia G J M, Woodson B T. The collapsing anatomical structure is not always the primary site of flow limitation in obstructive sleep apnea. Journal of Clinical Sleep Medicine. 2020; 16 (3):345-346. doi:10.5664/jcsm.8270
41. Vroegop A V, Vanderveken O M, Boudewyns A N, et al. Drug-induced sleep endoscopy in sleep-disordered breathing: Report on 1,249 cases. Laryngoscope. 2014; 124 (3):797-802. doi:10.1002/lary.24479

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A method for pharyngeal phenotyping in obstructive sleep apnea, comprising:
receiving manometry data for a subject;
extracting a plurality of features from the manometry data, wherein the extracted features comprise one or more of a high-level breath feature, a frequency feature, or a largest negative connected component (LNCC) feature;
inputting the extracted features into a trained machine learning model; and
predicting, using the trained machine learning model, at least one of a location of pharyngeal collapse for the subject or a degree of pharyngeal collapse for the subject.

2. The method of claim 1, wherein the high-level breath feature comprises a measure of manometry values across a plurality of pressure sensors of a manometry instrument.

3. The method of claim 1, wherein the frequency feature comprises a measure of a rate of change of manometry values per pressure sensor of a manometry instrument over the course of a breath.

4. The method of claim 1, wherein the LNCC feature comprises a measure of a shape or structure of a negative pressure envelop within an inspiration across a plurality of pressure sensors of a manometry instrument.

5. The method of claim 1, wherein the location of pharyngeal collapse and the degree of pharyngeal collapse are predicted using the trained machine learning model.

6. The method of claim 1, wherein the location of pharyngeal collapse is defined by a level of pharyngeal collapse.

7. The method of claim 6, wherein the level of pharyngeal collapse is the subject's velum, oropharynx, or hypopharynx.

8. The method of claim 1, wherein the location of pharyngeal collapse is defined by an anatomic structure.

9. The method of claim 8, wherein the anatomic structure is the subject's soft palate, oropharyngeal lateral walls, tongue base, or epiglottis.

10. The method of claim 1, wherein the degree of pharyngeal collapse is defined by a pharyngeal phenotyping classification system.

11. The method of claim 10, wherein the pharyngeal phenotyping classification system is the VOTE (velum, oropharynx, tongue base, or epiglottis) classification system.

12. The method of claim 1, further comprising:
receiving patient data associated with the subject; and
extracting one or more features from the patient data, wherein the extracted features comprise the plurality of features extracted from the manometry data and the one or more features extracted from the patient data.

13. The method of claim 12, wherein the patient data comprises anthropomorphic data, demographic data, polysomnography (PSG) data, video data, or electronic medical record data.

14. The method of claim 1, further comprising generating display data for the location of pharyngeal collapse.

15. The method of claim 14, further comprising presenting the display data on a display device.

16. The method of claim 1, further comprising generating a text-based description of the location of pharyngeal collapse.

17. The method of claim 16, further comprising presenting the text-based description on a display device.

18. The method of claim 1, further comprising generating a heatmap image associated with a breath from the manometry data.

19. The method of claim 18, further comprising presenting the heatmap image on a display device.

20. The method of claim 18, further comprising:
selecting a representative breath from the manometry data and a corresponding heatmap image associated with the representative breath; and
presenting the corresponding heatmap image on a display device.

21. The method of claim 1, wherein the trained machine learning model is a supervised machine learning model or a semi-supervised learning model.

22. The method of claim 1, wherein the trained machine learning model is a K-nearest neighbor (k-NN) classifier.

23. The method of claim 1, wherein the trained machine learning model is a support vector machine classifier, a logistic regression classifier, a random forest classifier, or a Naïve Bayes classifier.

24. A method, comprising:
performing pharyngeal phenotyping in obstructive sleep apnea according to claim 1; and
recommending the subject for an intervention based on the at least one of the location of pharyngeal collapse or the degree of pharyngeal collapse predicted by the trained machine learning model.

25. The method of claim 24, further comprising performing the intervention on the subject.

26. The method of claim 24, wherein the intervention includes implanting a medical device in the subject, the medical device being configured to deliver nerve stimulation therapy.

27. The method of claim 24, wherein the intervention is a surgical, medical, or device-based intervention.

28. A method, comprising:
receiving manometry data for a subject;
extracting a plurality of features from the manometry data for the subject, wherein the extracted features comprise one or more of a high-level breath feature, a frequency feature, or a largest negative connected component (LNCC) feature;
inputting the extracted features into a trained machine learning model; and
predicting, using the trained machine learning model, whether the subject is a favorable or non-favorable candidate for an intervention.

29. A system for pharyngeal phenotyping in obstructive sleep apnea, comprising:
at least one processor; and
a memory operably coupled to the at least one processor, the memory having computer-executable instructions stored thereon that, when executed by the at least one processor, cause the at least one processor to:
receive manometry data for a subject;
extract a plurality of features from the manometry data for the subject, wherein the extracted features comprise one or more of a high-level breath feature, a frequency feature, or a largest negative connected component (LNCC) feature;
input the extracted features into a trained machine learning model; and
receive, from the machine learning model, at least one of a location of pharyngeal collapse for the subject or a degree of pharyngeal collapse for the subject.

30-59. (canceled)
